(19) United States
(12) Patent Application Publication
(10) Pub. No.: US 2022/0364139 A1
(43) Pub. Date: Nov. 17, 2022

(54) METHODS FOR DETERMINING GROWTH AND RESPONSE

(71) Applicant: PATAIGIN, LLC, Baltimore, MD (US)

(72) Inventors: Matthew SORENSEN, Seattle, WA (US); Erik NILSSON, Seattle, WA (US)

(21) Appl. No.: 17/762,332

(22) PCT Filed: Sep. 24, 2020
(86) PCT No.: PCT/US2020/052562

§ 371 Date: Mar. 21, 2022

Related U.S. Application Data
(60) Provisional application No. 62/905,361, filed on Sep. 24, 2019.
(60) Provisional application No. 62/956,438, filed on Jan. 2, 2020.

(51) Int. Cl. C12Q 1/18; C12Q 1/04; C12Q 1/06

(52) U.S. Cl. CPC C12Q 1/18; C12Q 1/04; C12Q 1/06

(57) ABSTRACT

Methods for processing and analyzing samples are presented, said methods useful for, among other uses, analysis of lipids and other analytes of a sample, said methods further useful for identification of microorganisms at the level of species, identification of microorganisms at a level other than species, detecting infections and other diseases, detecting and measuring growth of an organism, detecting and measuring an environmental response of an organism, determining and/or measuring antimicrobial resistance of a microorganism, and for other purposes.

### BACKGROUND

The present invention relates at least to the fields of analytical chemistry, microbiology, data analysis, and medicine. More particularly, the present invention relates at least to the technical fields of diagnostic medicine and microbial assays.

### BRIEF DESCRIPTION OF THE DRAWINGS

FIG. 1 shows spectra for samples of a P. fluorescens sample grown in a range of kanamycin concentrations with D₂O.

FIG. 2 shows a graph of the ratio of heavy/light LA ions from the spectra in FIG. 1.

FIG. 3 shows a mass spectrum of a lipid extract of E. coli.

FIG. 4 shows synthetic [M−H]-mass spectra generated from PG and PE structures.

FIG. 5 shows a summary synthetic spectrum integrated from various PG and PE structures, also shown.

FIG. 6 shows a summary synthetic spectrum compared to an actual MALDI mass spectrometry lipid spectrum from a lipid extract of E. coli.

FIG. 7 shows the spectra from the previous figure with labeled masses.

FIG. 8 shows a mass spectrum of a lipid extract of deuterated E. coli.

FIG. 9 shows a mass spectrum of a lipid extract of deuterated E. coli and a mass spectrum of a lipid extract of E. coli that is not deuterated.

FIG. 10 shows synthetic [M−H]-mass spectra generated from PG and PE structures with deuterated fatty acid chains.

FIG. 11 shows a summary synthetic spectrum integrated from various deuterated PG and PE structures, also shown.

FIG. 12 shows a summary synthetic spectrum compared to an actual MALDI mass spectrometry lipid spectrum from a lipid extract of deuterated E. coli.

FIG. 13 shows the spectra from the previous figure with labeled masses.

FIG. 14 shows a spectrum from a lipid extract of E. coli incubated in a deuterium-enriched environment where growth was mechanically retarded compared to a spectrum from a lipid extract of E. coli grown in an environment with normal hydrogen isotope distribution.

FIG. 15 shows a mass spectrum of a lipid extract of deuterated E. coli and a mass spectrum of a lipid extract of E. coli that is not deuterated.

FIG. 16 shows a detail of a mass spectrum of a lipid extract of deuterated E. coli and a mass spectrum of a lipid extract of E. coli that is not deuterated.

FIG. 17 shows a detail of a mass spectrum of a lipid extract of deuterated E. coli and a mass spectrum of a lipid extract of E. coli that is not deuterated.

FIG. 18 shows a detail of a mass spectrum of a lipid extract of deuterated E. coli and a mass spectrum of a lipid extract of E. coli that is not deuterated.

FIG. 19 shows a detail of a mass spectrum of a lipid extract of deuterated E. coli and a mass spectrum of a lipid extract of E. coli that is not deuterated.

FIG. 20 shows a mass spectrum of a lipid extract of deuterated E. coli, a mass spectrum of a lipid extract of E. coli that is not deuterated, and a mass spectrum of a lipid extract of mixture of deuterated and undeuterated E. coli.

FIG. 21 shows a detail of a mass spectrum of a lipid extract of deuterated E. coli, a mass spectrum of a lipid extract of E. coli that is not deuterated, and a mass spectrum of a lipid extract of mixture of deuterated and undeuterated E. coli.

FIG. 22 shows a detail of a mass spectrum of a lipid extract of deuterated E. coli, a mass spectrum of a lipid extract of E. coli that is not deuterated, and a mass spectrum of a lipid extract of mixture of deuterated and undeuterated E. coli.

FIG. 23 shows a detail of a mass spectrum of a lipid extract of deuterated E. coli, a mass spectrum of a lipid extract of E. coli that is not deuterated, and a mass spectrum of a lipid extract of mixture of deuterated and undeuterated E. coli.

FIG. 24 shows a detail of a mass spectrum of a lipid extract of deuterated E. coli, a mass spectrum of a lipid extract of E. coli that is not deuterated, and a mass spectrum of a lipid extract of mixture of deuterated and undeuterated E. coli.

FIG. 25 shows spectra from a times series of E. coli growth.

FIG. 26 shows a comparison of lipid A ion heavy/light ratio vs. growth time for E. coli.

FIG. 27 shows a comparison of mass spectra extracted using the method of embodiment 1d to a spectrum extracted using a method of embodiment 1a.

FIGS. 28A, 28B, and 28C show some types of membrane lipids that are extracted from microbes using at least one embodiment of the present invention, and an example structure for each type.

FIG. 29 shows an example structure of lipid A from that in at least one embodiment of the present invention is extracted from Escherichia coli and an [M−H]-ion observed via mass spectrometry at a mass and/or m/z value of about 1797 Da.

FIG. 30 shows a Raman spectrum of deuterated E. coli.

FIG. 31 shows a Raman spectrum of deuterated E. coli by wavenumber.

It should be noted that the figures herein are not necessarily to scale. Some spectra are shown inverted and/or offset from the baseline, while others are not; this has no significance other than for clarity in comparing similar spectra.

### SUMMARY

The present disclosure comprises in at least one aspect one or more methods for making an identification, measurement, and/or estimate of the antimicrobial resistance of microorganisms in a sample to one or more antimicrobial agent, antimicrobial class, or antimicrobial combination. In at least one embodiment, the aforementioned identification, measurement, and/or estimate is used to diagnose an infectious disease and/or determine whether a microbe is resistant or susceptible to an antimicrobial agent that may be used to treat a patient.

Infectious diseases remain a serious health burden throughout the world. Diagnostic tests are critical to treating infectious diseases, but current tests are slow and lack sensitivity. Faster, more sensitive, and/or more accurate tests would allow clinicians to give the right treatment to the patient sooner.

In at least one embodiment, microbial membrane lipids such as phospholipids are evaluated to determine microbial growth. These lipids are abundant in microbial membranes and can extracted by methods known to those skilled in the art as well as methods described herein. Therefore, measuring microbial membrane lipids allows for sensitive determination of microbial growth.

Hydrogen comprises two main isotopes: ordinary ¹H hydrogen (“H”) and ²H hydrogen “deuterium,” or “D”). Normally, water contains about 0.0115% deuterium, but deuterium may be artificially added or removed from water. The differences in reaction rates for exchange with H vs. D in solution become increasing small as the ratio between ¹H and D (“H-D ratio”) in solution becomes less extreme. Thus for at least one embodiment of the present invention, the H-D ratio of at least one solution is between about 200:1 and 4:1 or equivalently between about 0.5% and 20% D molar concentration, and exchangeable hydrogens on pathway intermediaries such as for example phospholipid synthesis intermediaries substantially equilibrate to an H-D ratio that is strongly correlated with the H-D ratio of the surrounding solution. As a further example without limiting the present invention, NADPH (nicotinamide adenine dinucleotide phosphate) rapidly equilibrates to an H-D concentration approximately equal to the solution H-D ratio in many living cells (Zhang 2017 DOI: 10.1021/jacs.7b08012).

Herein, depending on context, “hydrogen” or “H” means either ¹H as distinct from ²H, a chemical mixture of ¹H and ²H with an H-D ratio that is unknown, a chemical mixture of ¹H and ²H with the natural H-D abundance ratio, or some other ratio, or an individual or exemplar atom that may be either ¹H or D with a probability that is unknown, equal to the natural H-D abundance ratio, or equal to some other ratio.

Herein, “deuterated” applied to molecules should be understood, unless context indicates otherwise, to mean equally a molecule that has had every possible H replaced by a D, but also a molecule where Hs have been replaced by Ds at a ratio or frequency above that of the natural abundance of Deuterium. Herein, “undeuterated” applied to molecules should be understood, unless context indicates otherwise, to mean equally a molecule that has not had any H replaced by a D, but also a molecule with an H-D ratio equal to or with a probable H-D ration equal to that of the natural abundance of deuterium. Herein “deuterated” applied to water or a solution or other material containing water means that the amount of deuterium is at a level higher than the natural abundance of deuterium. Herein “undeuterated” applied to water or a solution or other material containing water means that the amount of deuterium is at a level equal to or lower than the natural abundance of deuterium.

Some biological hydrogens do not exchange with solution, such as for example hydrogens bound to carbon do not ordinary exchange with solution. For example, and without limiting the disclosure, hydrogens that are part of a carbon chain of a lipid will generally not equilibrate to the H-D ratio of the surrounding solution. As a further example without limiting the present invention, the number of ¹H and D atoms on the straight chain portions of PC (phosphatidylcholine) molecules are not affected by the H-D ratio in the surrounding solution, nor is the number of the aforementioned ¹H and D atoms affected by one or more changes in the aforementioned H-D ratio.

Further considering the aforementioned pathways, in some cases the H-D ratio of precursor materials, media or food, or similar can also be altered, and said H-D ratio or ratios can cause an altered H-D ratio in pathway products. Consequently, for any method of the present invention using an altered H-D ratio in solution, one or more methods use altered H-D ratios of media or some other substance in an organism's environment, instead of, or in addition to the H-D ratio of water.

Deuterium or D₂O is significantly less expensive than other isotope enrichment alternatives, isotope enrichment of solution is less expensive and more convenient than enrichment of media, and H-D exchange with water in solution has low hysteresis. In a preferred embodiment, solution is enriched with D or equivalently D₂O. Further, at least one embodiment achieves deuterium enrichment by adding water enriched with D₂O to microbial growth media.

As is known to those skilled in the art, D concentrations of more than 30% can affect microbial growth rates. Thus, in at least one embodiment, solution D concentration is less than 30%. As solution D concentration decreases, the average number of D atoms per phospholipid decreases, and the distribution of deuterated lipid peaks in a mass spectrum narrows, so that the most intense deuterated phospholipid peak in a mass spectrum increases in intensity with decreasing solution D concentration. For many common membrane phospholipids, at some D concentration less than 5%, each prominent deuterated ion has nearly the same mass as an undeuterated ion, making deuterated ions difficult to distinguish from undeuterated ions in a mass spectrum. At a very low solution D concentration, a significant fraction of phospholipids and other molecules are not deuterated at all. In at least one embodiment, at least one solution is used which has a D concentration of less than 30%, less than 10%, about 8%, less than 8%, between 8% and 5%, about 5%, between 5% and 3%, about 3%, or less than 3%. The aforementioned at least one solution provides the advantages of a relatively small amount of D required and thus low cost, little or no detectable effect on microbial growth rate, little or no detectable effect on lag time, and high signal intensity of D ion MS peaks resulting in advantageous sensitivity, but wherein nonetheless for a given MS system or technique, D concentration is still sufficient such that deuterated and undeuterated ions can be distinguished, preserving sensitivity. In at least one further embodiment, one or more deuterated peaks of an analyte are sensitively detected in a mass spectrum, and in at least one additional further embodiment, growth and/or antimicrobial resistance of a sample is detected and/or measured by detecting the aforementioned deuterated peaks and/or by quantitatively or qualitatively comparing the aforementioned deuterated peaks to undeuterated peaks of the deuterated analyte and/or another analyte in the same mass spectrum and/or a different mass spectrum from the aforementioned sample and/or a different sample. In at least one embodiment, deuterated ions are detected and/or measured via at least one of a parent ion spectrum, a tandem ion spectrum, a multistage mass spectrometry (MSⁿ) spectrum, mass spectrometry (MS) spectra of another kind, or a technique other than MS.

As described herein, embodiments include deuterated lipids or deuterated lipid mimetics such as lipooligosaccharide/lipid A-based mimetics. In addition, a lipid composition as described in U.S. Pat. No. 10,358,667 to Ernst et al., or a lipid composition as described in US Patent Publication 2020/0121705 to Ernst et al, each of which are incorporated herein by these references.

As described herein, embodiments of the present invention sensitively measure growth and determine antimicrobial susceptibility for one or more microbial species in 6 hours or less. In at least one embodiment, antimicrobial susceptibility is determined in a period longer than 6 hours for a fastidious or other organism such as for example Mycobacterium tuberculosis, an organism for which antibiotic susceptibility testing typically takes longer than 6 hours, or an organism for which antibiotic susceptibility testing is not typically done but would be expected by those skilled in the art to take longer than 6 hours.

The present invention relates to methods for processing one or more samples and/or components of samples, to ascertain or estimate facts about said samples, and/or to extract specific chemicals or classes of chemicals from said samples. In at least one embodiment, at least one sample is a biological sample. In at least one embodiment, said biological sample contains, may contain, and/or is suspected to contain at least one microbial species. In at least one embodiment, said a least one microbial species is one or more of a bacteria, archaea, fungi, or protozoa.

### Microbial Lipid Synthesis and Deuterium

In at least one embodiment, an organism is sequentially placed in or subjected to two different environments, wherein at least one component of said environments contains a different H-D ratio. For example, and without limiting the disclosure, a microbial sample is grown in a first environment that is undeuterated, then placed in a second environment containing deuterated water. The molecular products of one or more pathways are then interrogated via a method such as mass spectrometry (“MS”), Raman spectroscopy, and/or nuclear magnetic resonance spectroscopy (“NMR”), whereby elevated D levels in said molecular product are detected and indicate the operation of said one or more pathways during the time the bacteria was in the second environment.

In at least one embodiment, in one or more microorganisms or other organisms, growth or the absence of growth above some threshold amount, degree, or extent is detected and/or measured by detecting and/or measuring deuterated membrane molecules using any appropriate method described in this application.

The present invention comprises in at least one aspect one or more methods for extracting lipids, proteins, and/or other molecules from samples.

In at least one embodiment, antibiotic susceptibility, antibiotic resistance, antimicrobial susceptibility, and/or antimicrobial resistance of one or more organisms is detected and/or measured, and/or a minimum inhibitory concentration (“MIC”) is measured, determined, and/or estimated by detecting and/or measuring deuterated membrane molecules using any appropriate method described herein. For example, and without limiting the disclosure, a microbial sample is grown in an environment containing at least one deuterated substance and an antimicrobial agent at some concentration. If the concentration of the antimicrobial agent is below the MIC for at least one microbial species, strain, or other population in the sample, the at least one microbial species, strain, or other population in the sample will grow, and growth is detected using any appropriate method described herein. As a further example without limiting the present invention, a microbial sample is divided and grown in multiple microwells containing deuterated solutions and also containing an antimicrobial agent at a range of concentrations in aforementioned multiple microwells. The growth rates of said microbial sample in said multiple microwells is estimated using any applicable method of this application. As antimicrobial concentration approaches the MIC of the microbial sample, the growth rate of the microbial sample approaches zero. Consequently, in at least one further embodiment, the MIC for the microbial sample and antimicrobial agent can be estimated from two or more concentrations of antimicrobial agent, so long as at least one said antibiotic concentration is less than the MIC, even if growth inhibition was not observed. As a further example without limiting the present invention, a bacterial sample is grown in one or more microwells containing a deuterated solution and containing an antimicrobial agent at a specific concentration in each of the one or more microwells. The growth rates of said microbial sample in said one or more microwells is estimated from measurements of the microwells multiple times, using any appropriate method of this application, using one or more measurements from one or more of the microwells at one or more times.

In at least one embodiment, the response of an organism, cell line, or other biological system to an environmental condition is assessed using any appropriate method of this application. For example, and without limiting the disclosure, the bacterial species Vibrio fischeri adds phosphoethanolamine to some of the lipid A in the lipopolysaccharide (“LPS”) of its membrane in response to low pH, conferring resistance to polymyxins. Further, many other Gram-negative bacteria modify their lipid A in response to environmental cues such as pH, temperature, and. aerobicity. Similar to PCs discussed above, lipid A precursors equilibrate to solution H-D ratio, but lipid A acyl chains do not. So, using any appropriate method herein, the H-D ratio of lipid A extracted from a bacterial membrane indicates the H-D ratio of surrounding solution when said lipid A was synthesized. Use of one or more H-D ratios or ratio changes combined with changes in environmental conditions then sensitively detects and/or measures the relationship between an environmental cue and a lipid A modification. Further, an H-D gradient or other concentration function combined with a gradient or other function in an environmental condition can be used to measure a specific trigger value of an environmental switch or otherwise interrogate the relationship between an environmental stimulus and a response.

Herein, where reference is made to an organism's interaction with its environment such as for example “environmental condition” or “environmental response,” the meaning of “environmental” includes all objects and effects from any and all of the natural world of nonliving things, living things of the same and/or different species or strains than the organism, the organism itself and its component parts, past and present effects of the organism itself, communities of organisms possibly including one or more communities of which the organism itself is a member, and the built world of artificially made things. That is, in the context of “environmental condition” or “environmental response,” the “environmental” influences on an organism means the totality of influences on that organism. Herein, unless context indicates otherwise, a “response” when used in reference to a organism should be understood to mean a response of an organism to stimuli, such as without limiting the invention in any way an environmental response of the aforementioned organism.

In at least one embodiment, lipids are extracted for analysis from one or more of microorganisms, bacteria, archaea, fungi, or protozoa. In at least one embodiment, lipids are extracted for analysis from the membrane of one or more organisms.

The present invention comprises in at least one aspect one or more methods for identifying or measuring the presence or quantity in a sample of one or more microbial species, one or more microbial taxa above the level of species, one or more microbial strains, and/or one or more mammalian or non-mammalian cell lines or cultures thereof.

The present invention comprises in at least one aspect one or more methods for identifying or measuring in a sample, for one or more microbial species, one or more microbial taxa above the level of species, and/or one or more microbial strains, one or more patterns or levels of antimicrobial susceptibility or resistance, virulence, and/or one or more other categories such as without limitation Gram stain.

The present invention comprises in at least one aspect one or more methods for estimating the quantity of one or more microbial species, one or more microbial taxa of species, and/or one or more strains in a sample.

The present invention comprises in at least one aspect one or more methods for diagnosing a microbial infection or other disease, and/or making estimates or predictions about the past, present, or future status of a disease.

The present invention comprises in at least one aspect one or more methods for determining and/or measuring one or more microbe's resistance or resistance potential to one or more antimicrobial agent, antimicrobial class, or antimicrobial combination.

The present invention comprises in at least one aspect one or more methods for making quantitative estimates from mass spectrometric data, and/or for using a mass spectrometer to measure the relative and/or absolute quantity of one or more substances in a sample.

The present invention comprises in at least one aspect one or more methods for constructing computational and/or machine learning models for making quantitative estimates, measurements, and/or predictions with regard to one or more mass spectrometry spectra. For example, and without limiting the disclosure, in one embodiment, a machine learning model uses a mass spectrum to estimate the ratio between deuterated and undeuterated lipids of one or more chemical classes. Embodiments of the invention use machine learning and/or computational models on mass spectrometry and/or other data for at least the following purposes: to identify a chemical species by isotopic and/or chemical bond variation patterns; to identify a form of life including without limitation a species, genus, family, Gram-stain status, phylum, or kingdom by a pattern of chemical species; and to detect and/or measure growth and/or another environmental response by a pattern of chemical species or by comparison of two or more patterns of chemical species, including without limitation comparing a pattern of deuterated chemical species to a pattern of undeuterated chemical species.

Herein, unless specified otherwise or clear from context, “sensitivity” means the ability to identify a microbe from a comparatively small number of organisms in a sample. That is, high sensitivity implies a low limit of detection (“LOD”).

### Purposes for Measuring Growth and Response

In at least one embodiment of the present invention (“in at least one embodiment”), growth and/or response of an organism is detected and/or measured (“the analysis”) is performed for one or more of the following purposes.

In at least one preferred embodiment of the present invention, the analysis is performed for identification and/or quantification of microbes, and/or other purposes including without limitation the diagnosis, prognosis, and/or determination of antimicrobial resistance of microbes and/or microbial infections.

In at least one embodiment of the present invention, the analysis is performed for the purpose of identifying one or more microbe species and/or strains present in a sample. In at least one embodiment of the present invention, the analysis is performed for the purpose of identifying one or more microbes in a sample and/or for the purpose of determining and/or measuring the quantity of each and/or the quantity of all of said one or more microbes present in said sample.

In at least one embodiment of the present invention, the analysis is performed for the purpose of determining that microbes of one or more taxa are present in a sample, said one or more taxa having a taxonomic rank above species and a taxonomic rank that may be at least as high as the taxonomic rank of domain. In at least one embodiment of the present invention, the analysis is performed for the purpose of determining that microbes of one or more categories not corresponding to taxa, such as without limitation Gram stain, are present in a sample.

In at least one embodiment of the present invention, the analysis is performed for the purpose of determining and/or measuring antimicrobial resistance to an antimicrobial agent, antimicrobial class, or antimicrobial combination of one or more species, taxa, strains, heteroresistant clones, and/or other populations of microbes in a sample. In at least one embodiment of the present invention, the analysis is performed for the purpose of determining and/or measuring the virulence of a sample. In at least one embodiment of the present invention, the analysis is performed for the purpose of determining whether microbes are present above a threshold value of absolute number, concentration per unit volume, concentration per unit area, total mass, mass per unit volume or area, total microbial surface area, microbial surface area per unit volume or area, total microbial volume, or microbial volume per unit volume or area. In at least one embodiment, at least one said threshold concentration corresponds to at least one diagnostic criterion of at least one disease.

In at least one embodiment of the present invention, the analysis is performed for the purpose of determining that, below a threshold value of microbial concentration, number, mass, size, surface area, or other microbial measure, one or more microbes of a specific species and/or strain are not present in a sample. In at least one embodiment of the present invention, the analysis is performed for the purpose of determining that, below a threshold value of microbial concentration, number, mass, size, surface area, or other microbial measure, microbes of one or more taxa are not present in a sample, said one or more taxa having a taxonomic rank above species and a taxonomic rank that may be at least as high as the taxonomic rank of domain. In at least one embodiment of the present invention, the analysis is performed for the purpose of determining that, below a threshold value of microbial concentration, number, mass, size, surface area, or other microbial measure, microbes of one or more categories not corresponding to taxa, such as without limitation Gram stain, are not present.

In at least one embodiment of the present invention, the analysis is performed for the purpose of determining that, one or more or all microbial taxa, strains, and/or other populations in a sample grow and/or exhibit growth above a threshold value expressed as microbial concentration, number, mass, size, surface area, or another measure. In at least one embodiment of the present invention, the analysis is performed for the purpose of determining that, one or more or all microbial taxa, strains, and/or other populations did not grow and/or did not exhibit growth above a threshold value expressed as microbial concentration, number, mass, size, surface area, or other measure.

In at least one embodiment of the present invention, the analysis is performed for the purpose of discovering, identifying, and/or measuring a cooperative, antagonistic, or other communication or response between organisms, a communal antimicrobial attack, a communal antimicrobial response, a biofilm matrix effect, a community antibiogram, and/or a similar multi-species, multi-strain, or multi-clone behavior.

In at least one embodiment of the present invention, the analysis is performed for the purpose of discovering, identifying, and/or measuring a specific substance and/or target with potential antimicrobial properties.

In at least one embodiment of the present invention, the analysis is performed for the purpose of refining lipids from a sample or other source for use in a product such as a chemical reagent, chemical marker, affinity reagent or target or component or source of same thereof, drug, drug product, drug ingredient, fuel, or any other product.

Herein, “strain” means any genetic and/or phenotypic variation in an organism below the taxonomic rank of species, including without limitation specific mutations whether previously known or unknown, differences in gene product expression levels, clones, adaptations to growth conditions, variations related to pathogenicity such as without limitation virulence and antibiotic resistance, and/or different life phases of an organism even if not usually thought of as distinct strains. In cases where there is not general agreement on groupings into species, strains, and/or other taxa, “strain” additionally means those organisms that would, in at least one relevant context, be considered to comprise one species, but which are biologically distinct in at least one other relevant context.

### DETAILED DESCRIPTION

The present disclosure relates to methods for processing one or more samples and/or components of samples, to ascertain or estimate facts about said samples, and/or to extract specific chemicals or classes of chemicals from said samples. In at least one embodiment, at least one sample is a biological sample. In at least one embodiment, said biological sample contains, may contain, and/or is suspected to contain at least one microbial species. In at least one embodiment, said a least one microbial species is one or more of bacteria, archaea, fungi, or protozoa.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, is to be understood to include any integer within the recited range, unless otherwise indicated. As used herein, the term “about” means±20% of the indicated range, value, or structure, unless otherwise indicated. As used herein, the term “about” as applied to mass spectra m/z values means the greater of ±20% or ±100 m/z. As used herein, the term “about” as applied to CFU/mL (colony forming units/mL) means±10^{0.5}, equivalent to multiplying or dividing the concentration by a factor of approximately 3.16. As used herein, the term “about” as applied to liquid volume means the greater of ±20% or ±0.5 mL. It should be understood that the terms “a” and “an” as used herein refer to “one or more” of the enumerated components. The use of the alternative (e.g., “or”) should be understood to mean either one, both, or any combination of the alternatives. As used herein, the terms “include,” “have,” and “comprise” are used synonymously, which terms and variants thereof are intended to be construed as non-limiting.

“Optional” or “optionally” means that the subsequently described element, component, event, or circumstance may or may not occur, and that the description includes instances in which the element, component, event, or circumstance occurs and instances in which they do not.

In addition, it should be understood that the individual constructs, or groups of constructs, derived from the various combinations of the structures and subunits described herein, are disclosed by the present application to the same extent as if each construct or group of constructs was set forth individually. Thus, selection of particular structures or particular subunits is within the scope of the disclosure.

The term “consisting essentially of” is not equivalent to “comprising” and refers to the specified materials or steps of a claim, or to those that do not materially affect the basic characteristics of a claimed subject matter. For example, and without limiting the disclosure, a protein domain, region, or module (e.g., a binding domain, hinge region, or linker) or a protein (which may have one or more domains, regions, or modules) “consists essentially of” a particular amino acid sequence when the amino acid sequence of a domain, region, module, or protein includes extensions, deletions, mutations, or a combination thereof.

“Microbial organism,” “microbe,” or “microorganism” as used herein interchangeably refers to any one of a bacterial species; an archaea; a yeast and/or fungal species; or any microbial species other than bacteria, yeast, or fungi, e.g. protozoa. Herein, “fungus” and “fungi” refer to the kingdom Fungi in its entirety, even for example when fungi are considered in contrast to members of the Fungus kingdom such as yeasts. A microbial organism may exist as a single cell or in a colony of cells. As used herein, “organism” can refer to an intact and living organism, or an organism or components of an organism pre-processed such that they are or may not be living or comprise a whole or entire organism, such as without limitation fixation in formalin or ethanol. Further, the methods described herein can be applied equally to an organism that is a component or fragment of an organism, so long as the organism that is a component or fragment has, contains, or is a membrane. For example, and without limiting the disclosure, the methods described herein can be applied to membrane vesicles including outer membrane vesicles, vesicles of endomycorrhizal fungi, vesicles that are not extracellular vesicles, artificial vesicles, and other types of vesicles.

As used herein, “antimicrobial,” “antimicrobial agent,” and “treatment” are used interchangeably to mean any agent used to kill (microbicidal) or retard the growth of (biostatic) at least one microorganism. Antimicrobial medicines include, but are not limited to, antibiotics and antifungals.

As used herein, “membrane” includes any cellular membrane or membrane on a vesicle, including but not limited to outer membrane vesicles (OMVs), membrane vesicles (MVs), or any membrane fragment, or membrane raft.

It will be appreciated by those skilled in the art that an H-D ratio of a molecule will not ordinarily be in exact equilibrium with for example water solution, due to kinetic isotope effects. As used herein, “equilibrium” and “equilibrate” when used to refer to processes involving H-D exchange, processes involving exchange of deuterated and undeuterated molecules, or processes involving exchange of molecules with different degrees of deuteration, means respectively an approximate state of equilibrium or the process of reaching an approximate state of equilibrium.

One skilled in the art will appreciated that, just as ordinary water has a small percentage of deuterium, that a substance comprising approximately 100% D₂O will still include some amount of ¹H, and that just as some minimal H-D exchange always takes place in H₂O, some minimal H-D exchange takes place in any composition referred to as D₂O. Nonetheless, just as ordinary water allows for undeuterated molecules with specific properties, deuterated water allows for equivalent deuterated molecules with different specific properties. In each case, an unavoidable amount of the opposite isotope of hydrogen is always present at some level. Furthermore, water with intermediate H-D ratios will have intermediate properties and will often result in a mixture of H-D isotopologues for molecules with predictable and stable properties.

One skilled in the art will appreciate that H atoms exchange within a water solution and are mobile within the solution so that although those skilled in the art sometimes refer to a mixture of D₂O and H₂O, in such a mixture, the molecule DOH also occurs. Herein, where reference is made to a percentage of D₂O or a percentage of H₂O in a solution, this means the percentage of D or H respectively to all hydrogens bound or nominally bound to oxygen in the form of water, as if the solution had been formed from a mixture of the aforementioned percentage of pure D₂O combined with pure H₂O.

The following embodiments and descriptions describe embodiments and aspects of the disclosure, without limiting the disclosure in any way. Specific embodiments are enumerated and referred to as “EMBODIMENT A,” “EMBODIMENT 1,” etc. Also, certain embodiments provided by way of example for one or more embodiments or the like such descriptions are not referred to by letter or number.

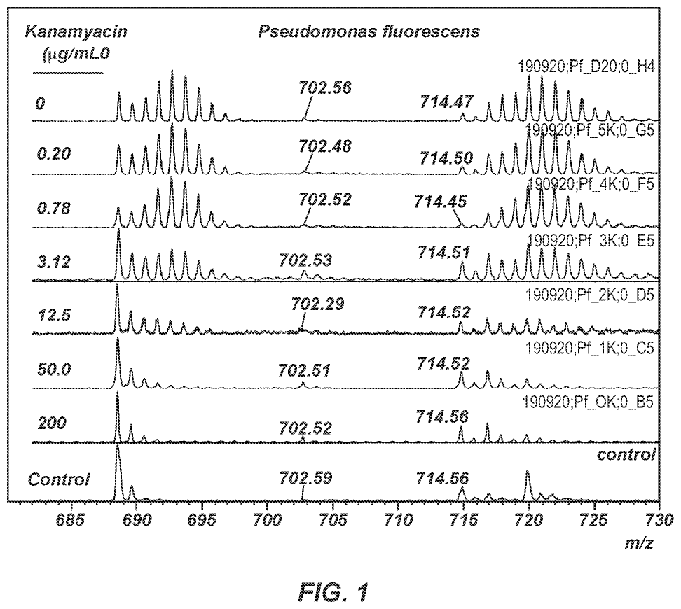

In FIG. 1 is shown eight MALDI mass spectra from about 680 to 730 m/z in negative ionization mode of membrane lipids extracted from a P. fluorescens strain that is susceptible to kanamycin. The aforementioned P. fluorescens strain was grown in seven environments containing Mueller-Hinton media and water containing 8% D₂O, with each of the seven environments also having a concentration of kanamycin from 0 μg/mL to 200 μg/mL. The same P. fluorescens strain was grown in ordinary water without added D₂O or kanamycin, shown in FIG. 1 as “Control.” The spectra from 200 μg/mL and 50 μg/mL kanamycin concentration have predominantly the same ion peaks as the Control spectrum, indicating that the growth of P. fluorescens was inhibited by kanamycin at these concentrations. At 12.5 μg/mL kanamycin, additional deuterated peaks are observed to the right of the peaks (greater m/z) corresponding to peaks observed in the aforementioned spectra, indicating that growth of P. fluorescens occurred at this concentration. At decreasing kanamycin concentrations below 12.5 μg/mL, deuterated peaks are observed with increasing intensity, indicating that growth increased as kanamycin concentration decreased.

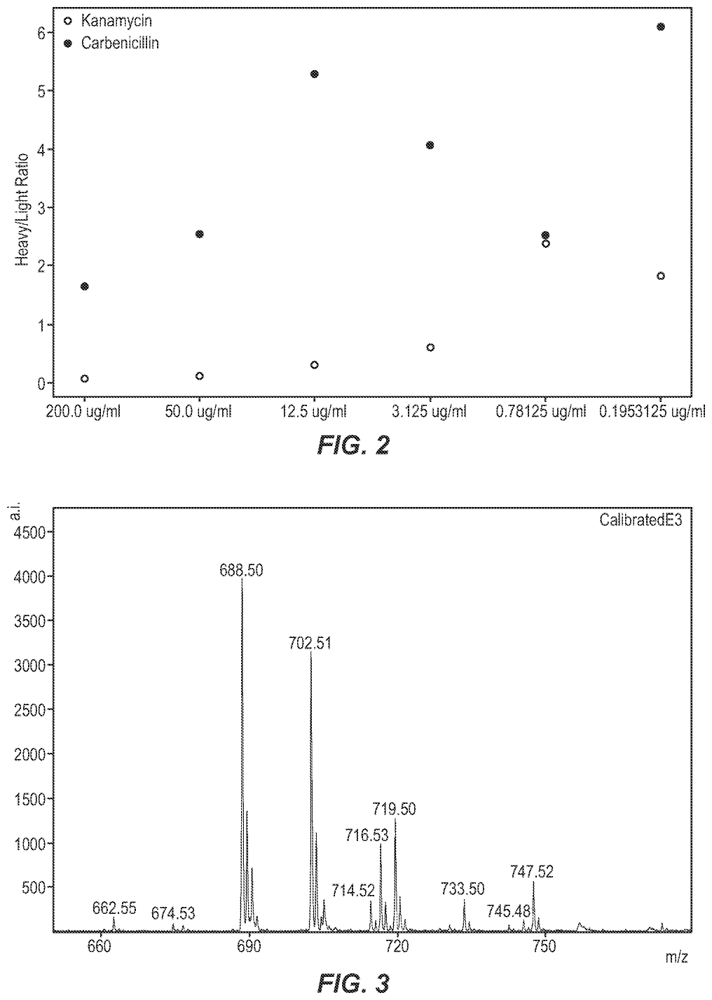

In FIG. 2 is shown a graph of the ratio of deuterated to undeuterated ions from the spectra in FIG. 1, showing that the ratio of heavy to light peaks can be used to determine MIC and to quantitatively estimate or measure growth in a sample and/or measure the growth inhibition caused by a concentration of an antibiotic. In at least one embodiment, a quantitative estimate as described above is used to estimate an MIC from one, two, or more than two antibiotic concentrations, or is used for some other method or purpose described herein, or for some other purpose. In at least one embodiment, electrospray ionization mass spectrometry or another type of mass spectrometry is used. In at least one embodiment, tandem mass spectrometry, MSⁿ, multiple reaction monitoring, or another fragmentation strategy and/or mass spectrometry technique is used. In at least one embodiment, mass spectrometry is not used, or is used in conjunction with another type of detection.

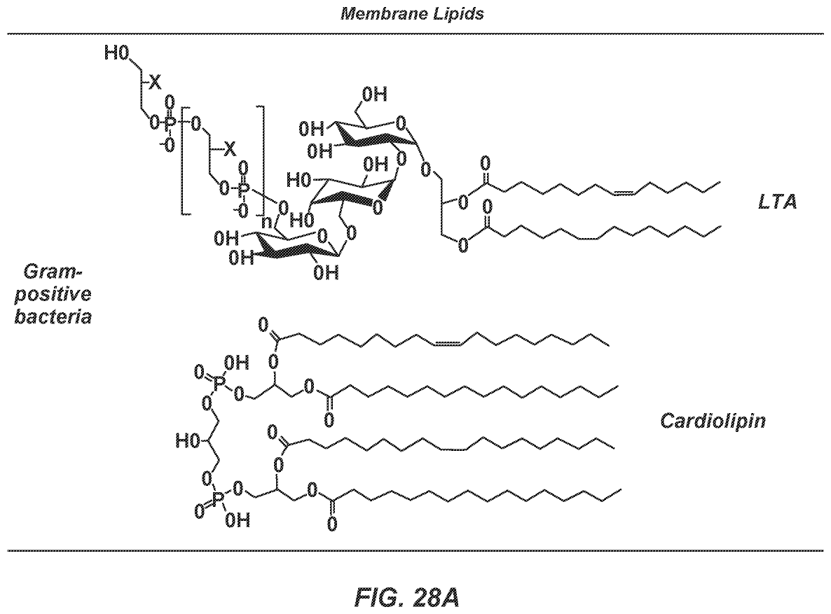

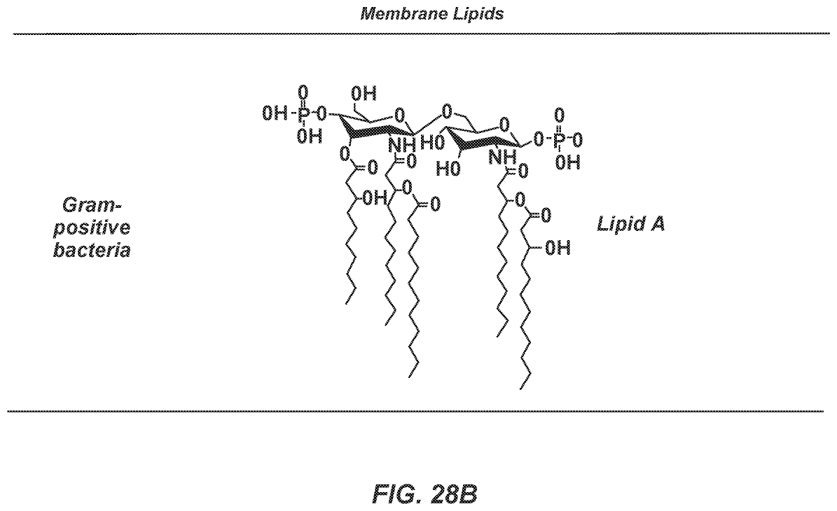

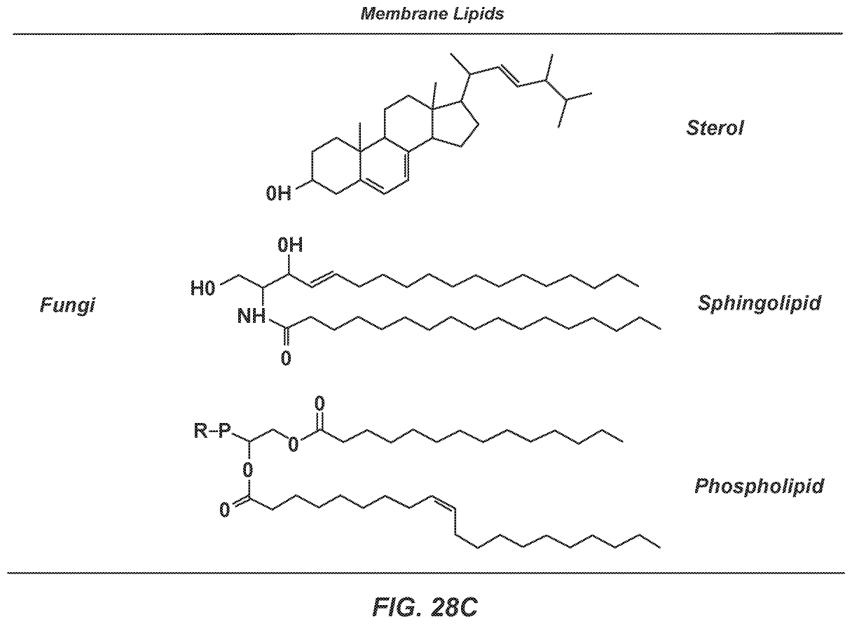

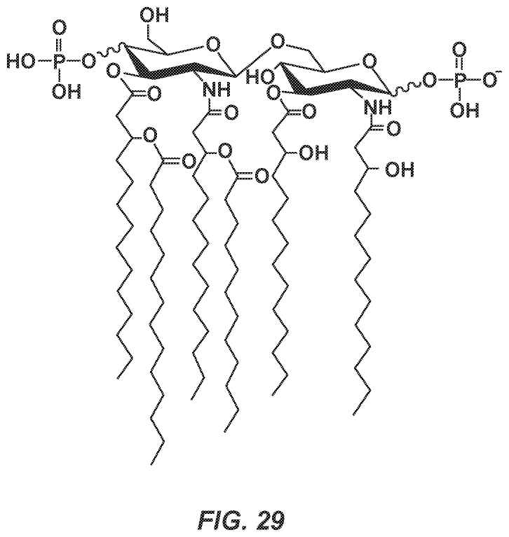

Lipid A (LA) the endotoxic portion of lipopolysaccharide (LPS) is embedded in the outer leaflet of the Gram-negative bacterial outer membrane. As an essential component of Gram-negative bacterial membranes, LA exhibits species-specific structural diversity. The general structure consists of a backbone of two glucosamine residues present as a B-(1-6)-linked dimer. This backbone can be diversified in response to specific environmental signals or between bacterial species. Specifically, changes in the fatty acid content varying both in the length and number of fatty acid side chains (e.g. tetra- to hepta-acylated) and phosphorylation patterns can differ as well. Additional modifications of the phosphate residues by monosaccharides, such as aminoarabinose or galactosamine and phosphoethanolamine can occur. The diversity of such species and environmentally-driven structural modifications are an adaptive mechanism that increases bacterial survival often through increasing resistance to host antimicrobial peptides, or in the avoidance of the host innate immune system. Precursor molecules (i.e.: molecules from which LA is cleaved during isolation) to LA include, but are not limited to LPS. Representative LA structures are shown in FIGS. 28 and 29.

Lipoteichoic acid (LTA) is a major cell wall component of Gram-positive bacteria. The Gram-positive cell wall is composed of cross-linked peptidoglycan variably decorated with teichoic acid polymers. Teichoic acid polymers are also linked to plasma membrane phospholipids. The general structure of LTA varies between species consisting of 2 or 4 acyl groups, of variable chain length. LTA from low G+C subdivisions of Gram-positive bacteria contains two fatty acid tails, while those from high G+C bacteria contain 4 fatty acid tails. Additionally, LTA can be variably modified with alanine (in response to low pH), or glycosyl linkages depending on bacterial background. Glycosyl linkages can include glycerol phosphate, galactose, or N-acetyl-glycerol. A representative LTA structure is shown in FIG. 28.

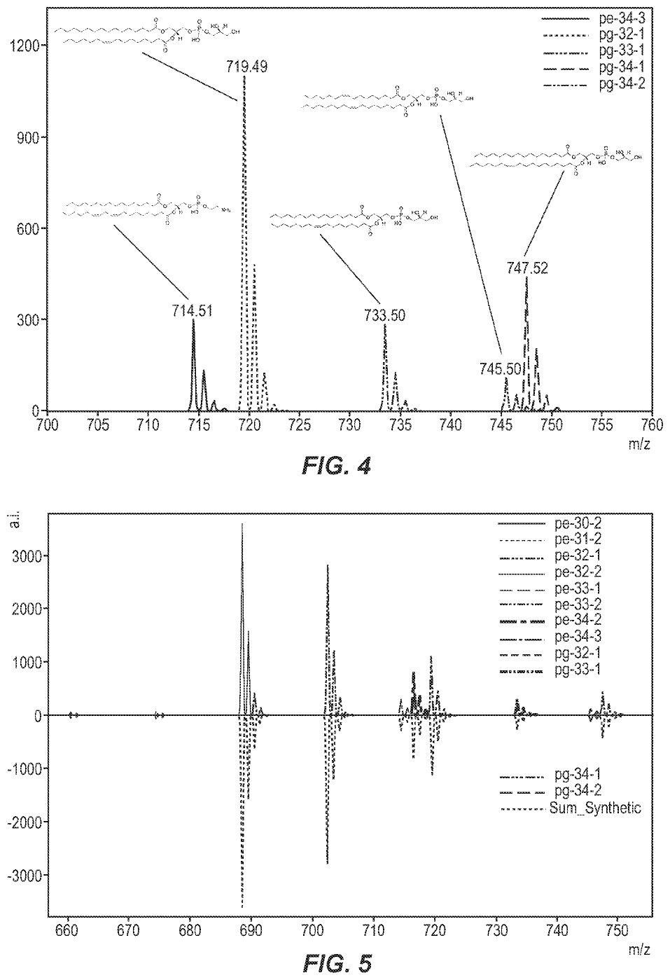

Phospholipids such as phosphatidylcholines (“PCs”), phosphatidylethanolamines (“PEs”), phosphoglycerols (“PGs”) and cardiolipins (“CLs”) are common microbial cell wall components. Representative PE and PG structures and ion peaks corresponding to said representative PE and PG structures as exemplar structures are shown in FIG. 4. A representative CL structure is shown in FIG. 28.

In at least one embodiment, a sample is processed, said sample being one or more of a culture plate colony or smear, a broth culture sample, a blood culture sample, a sample from a biofluid, a clinical sample, or a nonclinical sample. In at least one embodiment, a sample is one or more of an environmental sample, a veterinary sample, an agricultural sample, a food or food safety sample, an industrial sample, a process control sample, or a forensic sample. In at least one embodiment, the aforementioned biofluid is a biofluid from a human or non-human source. In at least one embodiment, a sample is processed, said sample comprising or derived from a urine specimen, a blood sample, a sample incubated in a blood bottle, sputum, a sample obtained from sputum, feces, wound effluent, mucus, buccal swab, nasal swab, vaginal swab, nipple aspirate, sweat, saliva, semen or ejaculate, synovial fluid, bronchoalveolar lavage, endotracheal aspirate, tears, a urinary catheter sample, a culture plate, or another clinical or medical sample, or another human or mammalian material. In at least one embodiment, a sample comprises a substance or assembly containing at least one component that is a sample as described herein.

The sample can be used as obtained, or can be processed in any way suitable for use with the methods of this disclosure. In one embodiment, the methods comprise analysis of microbes directly from a complex sample (i.e., no requirement for amplifying microbes present in the sample). In another embodiment, microbes are isolated from the sample, such as by streaking onto solid bacterial culture medium, followed by growth for an appropriate period of time.

In at least one embodiment, antimicrobial resistance to an antimicrobial agent of a microbial sample is evaluated by adding to at least one volume containing the antimicrobial sample at least the antimicrobial agent present in at least one concentration, growth media, and a liquid solution with an enriched concentration of deuterium oxide (D₂O). At appropriate conditions familiar to those skilled in the art, if the microbial sample is resistant to the antimicrobial agent, it will grow, but if the microbial sample is susceptible to the antimicrobial agent, it will not grow or grow to only a small degree.

In at least one embodiment one or more microbial species, taxa above the level of species, strain, clone, or other subpopulation in a sample is identified and furthermore the antimicrobial susceptibility of organisms in the sample to one or more antimicrobial agent, antimicrobial class, or antimicrobial combination is determined. In at least one further embodiment, identification and susceptibility determination are performed sequentially in any order, or are performed simultaneously. In at least one embodiment, the aforementioned identification includes identification of the presence or absence of at least one antimicrobial phenotype, such as for example addition of phosphoethanolamine and/or aminoarabinose to lipid A, which as those skilled in the art will appreciate in Gram-negative bacteria confers antimicrobial resistance to antimicrobial agents such as colistin.

Considering synthesis pathways such as for example lipid synthesis pathways in an organism such as for example a microorganism, at one or more points in said synthesis pathways, hydrogen (H) is exchanged between the surrounding water solution and one or more pathway intermediary molecular compounds (“pathway intermediaries”). However, H atoms on carbon straight chains are not exchanged with the surrounding solution. Thus, molecules synthesized in enriched D₂O concentration will have enriched deuterium (D) concentrations compared to the same molecules synthesized previously in the same sample before addition of D₂O or in an equivalent sample without D₂O enrichment.

Continuing with the description of the at least one embodiment, after a period of time to allow for possible growth, the microbial sample is evaluated by mass spectrometry (MS). If the microbial sample is resistant to the antimicrobial agent, growth will have occurred, and at least one molecule such as for example a membrane phospholipid will exhibit a pattern of ion peaks characteristic of D enrichment. However, if the microbial sample is resistant to the antimicrobial agent, no or minimal growth will have occurred, and molecules will not exhibit patterns characteristic of D enrichment. In one or more embodiment, the aforementioned approach is extended using methods familiar to those skilled in the art, to make quantitative estimates of growth from the ratio of deuterated to undeuterated peaks and/or to evaluate multiple molecules to improve measurement accuracy. In at least one embodiment, antimicrobial agents at multiple concentrations and/or multiple time points are evaluated to determine a minimum inhibitory concentration, and/or to test for antimicrobial susceptibility.

In various non-limiting embodiments, the methods in the present disclosure can be used to identify, determine the antimicrobial resistance of, identify an antimicrobial phenotype of, and/or analyze by any method described herein one or more bacteria (or sub-species thereof) including but not limited to Escherichia coli, Staphylococcus aureus, Staphylococcus epidermidis, Streptococcus pneumoniae, S. mitis, Streptococcus pyogenes, Stenotrophomonas maltophila, Mycobacterium tuberculosis, Neisseria gonorrhoeae, Neisseria meningitidis, Bordetella pertussis, B. bronchioseptica, Enterococcus faecalis, Salmonella typhimurium, Salmonella choleraesuis, Klebsiella pneumoniae, Pseudomonas aeruginosa, Acinetobacter baumannii, A. calcoaceticus, Bacteroides nordii, B. Salversiae, Enterobacter Subspecies including E. asburiae, E. cloacae, E. hormaechei, E. kobei, E. ludwigii, and E. nimipressuralis, extended spectrum B-lactamase organisms, as well as bacterium in the genus Acinetobacter; Actinomyces, Bacillus, Bacteroides, Bordetella, Borrelia, Brucella, Clostridium, Corynebacterium, Campylobacter, Deinococcus, Escherichia, Enterobacter, Enterococcus, Erwinia, Eubacterium, Flavobacterium, Francisella, Gluconobacter, Helicobacter; Intrasporangium, Janthinobacterium, Klebsiella, Kingella, Legionella, Leptospira, Mycobacterium, Moraxella, Neisseria, Oscillospira, Proteus, Pseudomonas, Providencia, Rickettsia, Salmonella, Staphylococcus, Shigella, Spirillum, Streptococcus, Stenotrophomonas Treponema, Ureaplasma, Vibrio, Wolinella, Wolbachia, Xanthomonas, Yersinia, and Zoogloea.

The various embodiments described herein can be combined to provide further embodiments.

### Embodiment A: Samples

In at least one embodiment, a sample is processed, said sample being one or more of a culture plate colony or smear, a broth culture sample, a blood culture sample, a sample from a biofluid, a clinical sample, a nonclinical sample, or some other sample. In at least one embodiment, a sample is one or more of an environmental sample, a veterinary sample, an agricultural sample, a food or food safety sample, an industrial sample, a biomanufacturing sample, a process control sample, or a forensic sample. In at least one embodiment, the aforementioned biofluid is a biofluid from a human or non-human source. In at least one embodiment, a sample is processed, said sample comprising or derived from a urine specimen, a blood sample, a sample incubated in a blood bottle, sputum, a sample obtained from sputum, urine, feces, wound effluent, mucus, buccal swab, nasal swab, vaginal swab, nipple aspirate, sweat, saliva, semen or ejaculate, synovial fluid, bronchoalveolar lavage, endotracheal aspirate, tears, a urinary catheter sample, a culture plate, or another clinical or medical sample, or another human or mammalian material. In at least one embodiment, a sample comprises a substance or assembly containing at least one component that is a sample as described herein.

In at least one embodiment, a sample is a biological sample. In at least one embodiment, said biological sample contains, may contain, and/or is suspected to contain at least one microbial species. In at least one embodiment, said at least one microbial species is a bacteria, archaea, fungi, or protozoa.

In at least one embodiment, a sample is a cell culture or material from a cell culture. For example, and without limiting the disclosure, in at least one embodiment, a sample is from a culture of a mammalian or non-mammalian cell line, a functional or other assay or read-out making use of a cell line, a bioreactor, a therapeutic cell culture, and/or an experimental cell culture.

### Embodiment B: Clinical Samples

In at least one embodiment, the aforementioned at least one sample is derived from a clinical specimen or sample. In at least one embodiment, at least one sample is obtained from a urine specimen, a blood sample, a sample incubated in a blood bottle, sputum, a sample obtained from sputum, feces, wound effluent, mucus, buccal swab, nasal swab, vaginal swab, nipple aspirate, sweat, saliva, semen or ejaculate, synovial fluid, bronchoalveolar lavage, endotracheal aspirate, tears, a urinary catheter sample, a culture plate, or another clinical or medical sample.

### Embodiment C: Non-Clinical Samples

In at least one embodiment, the aforementioned at least one sample is an industrial sample, an environmental sample, an agricultural sample, a veterinary sample, a food sample, a forensic sample, a manufacturing process sample, a fermentation sample, a sterility sample, or some other sample.

### Embodiment D: Methods of Extraction

In at least one embodiment, lipids are extracted for analysis using one or more of the “fast lipid assay test” method, the “fast in-needle extraction” method, the “BACLIB” method considered as a lipid extraction method, the “Caroff” method, the Bligh-Dyer method, the Folch method, the hot phenol method, two-phase extraction, the TAO method (Liang 2018 doi.org/10.1021/acs.analchem.8b02611), and methods equivalent to or derived from these methods.

In at least one embodiment lipids are extracted using one or more of the methods of embodiments 1a-1e.

### Embodiment E: Analysis without Extraction

In at least one embodiment, lipids are analyzed without being extracted from a microbial membrane. For example, and without limiting the disclosure, in at least one embodiment, at least one type of spectrometry is applied to microbes growing in solution. As a further example, in at least one embodiment, at least one type of Raman spectrometry is applied to microbes growing in solution, and C—H bonds are distinguished from C-D bonds without necessarily extracting the molecules containing the aforementioned C—H and/or C-D bonds. Continuing with the example of Raman spectroscopy, one skilled in the art will appreciate that microorganisms as well as cells of types other than microorganisms produce multiple types of molecules containing C—H bonds, and Raman spectroscopy will does not typically distinguish to a significant degree between a C—H bond in a lipid and a C—H bond in a molecule that is not a lipid. However, one skilled in the art will also appreciate that lipids are a major component of microbial membranes, and furthermore said lipids typically have a larger number of C—H bonds per unit weight than other common microbial molecules such as many abundant proteins. Thus, said spectroscopic interrogation still predominantly interrogates bonds in lipids and still measures a signal that is predominantly generated by microbial or cellular growth.

### Embodiment F: Lipid Classes

In at least one embodiment, one or more instance of one or more of the classes phosphatidylcholines (“PCs”), phosphatidylethanolamines (“PEs”), phosphoglycerols (“PGs”), lipopolysaccharide (“LPSs”), lipid As (“LAs”), sphingolipid (“SPs”), lipoteichoic acids (“LTAs”), and sterols (“STs”) is interrogated, extracted, modified, generated, and/or manipulated. The aforementioned lipid classes are provided by way of example without limiting the invention in any way.

### Embodiment 1A: Extraction of Lipids from a Sample

1. Place at least one sample as described in embodiments A-C or elsewhere herein on at least one location (“spot”) of a plate such as MALDI plate. Allow the at least one spot to dry or partially dry

2. Optionally, place a reagent or other material on the aforementioned spot. For example, and without limiting the disclosure, place 1 μL of a solution of citric acid and sodium citrate on a spot. Allow the sample to dry or partially dry.

3. Heat the plate. In at least one embodiment the plate is heated to about 110° C. for about 30 min. The plate is prevented from completely drying while it is heated by heating the plate in a humid atmosphere.

4. Wash the plate. In at least one embodiment, a plate is washed by at least one application of at least one liquid. Allow the plate to dry or partially dry.

One skilled in the art will appreciate that, in at least one embodiment the method of embodiment 1a extracts molecules that are not lipids in addition to or instead of extracting lipids. Regardless, to simplify explanation, embodiment 1a is herein referred to as a method for extracting lipids.

### Embodiment 1b: Extraction of Lipids from a Sample

1. Place at least one sample as described in embodiments A-C or elsewhere herein on at least one location (“spot”) of a plate such as a MALDI plate. Optionally allow the at least one spot to dry or partially dry.

2. Optionally, place a reagent or other material on the aforementioned spot. For example, and without limiting the disclosure, place 1 μL of a solution of citric acid and sodium citrate on a spot. Allow the sample to dry or partially dry.

3. Optionally dry the plate by heating it or by any other method known to those skilled in the art. In at least one embodiment the plate is heated to about 80° C. for about 5 min.

4. Apply on the aforementioned spot at least one solution containing at least one organic solvent or alcohol, optionally containing water, and optionally containing at least one MALDI matrix compound. In a preferred embodiment, the at least one solution is a mixture of chloroform-methanol-water at a ratio by volume of 12:6:1, to which 10 mg/mL of beta-carboline (also known as norharmane) is added. Allow the at least one sample to dry or partially dry.

Lipids are extracted from at least one microbial membrane into the solvent, whereupon they may be deposited on the surface of the aforementioned plate, and/or may crystalize with the at least one MALDI matrix compound. The method of this embodiment 1b extracts many membrane lipids that are typically extracted with methods of embodiments 1a, 1c, 1d, or 1e.

One skilled in the art will appreciate that, regardless of whether the aforementioned at least one solution contains a MALDI matrix, it is possible but not necessary to subsequently apply a MALDI matrix solution at a later step in preparing the sample for MALDI analysis. In at least one embodiment, subsequent to embodiment 1b, a MALDI matrix is or is not applied to the at least one MALDI spot.

One skilled in the art will appreciate that, in at least one embodiment the method of embodiment 1b extracts molecules that are not lipids in addition to or instead of extracting lipids. Regardless, to simplify explanation, embodiment 1b is herein referred to as a method for extracting lipids.

### Embodiment 1c: Extraction of Lipids from a Sample

1. Place a sample as described in embodiments A-C or elsewhere herein in a microchannel. In at least one embodiment the aforementioned microchannel is the bore of a needle as in or similar to a hypodermic needler or some other microchannel.

2. Optionally allow the sample to dry or partially dry. In at least one embodiment, at least one reagent or other material is pre-applied to at least one microchannel or a region of at least one microchannel before a sample is placed in said microchannel. For example, and without limiting the disclosure, place a solution of citric acid and sodium citrate in the bore.

3. Optionally heat the microchannel. In at least one embodiment, the microchannel is heated to less than 80° C., about 80° C., about 85° C., about 90° C., about 95° C., about 100° C., about 110° C., about 120° C., about 125°, about 130°, about 135° about 140°, or above 140° C. In at least one embodiment, the microchannel is heated for less than 5 min., about 5 min, about 7 min, about 10 min, about 15 min, about 20 min, about 25 min, about 30 min, or longer than 30 min.

In a preferred embodiment, at least one microchannel is prevented from completely drying while it is heated by heating the at least one microchannel in a humid atmosphere. In at least one embodiment, the at least one microchannel is heated in an acidic atmosphere. In at least one embodiment, the at least one microchannel is heated in an atmosphere containing or comprised of acetic acid vapor. In at least one embodiment, the at least one microchannel is prevented from partly or completely drying while it is heated by supplying moisture periodically or continuously to the bore.

4. Optionally, wash the at least one microchannel. In at least one embodiment, the at least one microchannel is washed by at least one application of at least one liquid. In at least one embodiment, at least one liquid is squirted through the at least one microchannel with a syringe. In at least one embodiment, at least one liquid is water. In at least one embodiment, at least one liquid is a mixture or solution containing at least one of water, a detergent, an alcohol, an emulsifier, and an organic solvent. After at least one application of at least one liquid, optionally allow the at least one microchannel to dry or partially dry.

One skilled in the art will appreciate that, in at least one embodiment the method of embodiment 1c extracts molecules that are not lipids in addition to or instead of extracting lipids. Regardless, to simplify explanation, embodiment 1c is herein referred to as a method for extracting lipids.

One skilled in the art will appreciate that at least one embodiment of this embodiment 1c is suitable for extracting lipids for a manufactured product or similar. One skilled in the art will appreciate that at least one embodiment of this embodiment 1c is suitable for extracting lipids for use with an electrospray mass spectrometer.

### Embodiment 1d: Extraction of Lipids from a Sample

Extract samples using the method of embodiment 1b, as follows: skip optional step 2 of embodiment 1b; perform step 3 of embodiment 1b by drying the plate at 80° C. for about 5 min; perform step 4 of embodiment 1b by applying 1 μL of a solution of chloroform-methanol-water at a ratio by volume of 12:6:1, to which 10 mg/mL of beta-carboline has been added.

### Embodiment 1e: Extraction of Lipids from a Sample

Extract lipids from a sample via two-phase extraction, or by some other method, yielding extracted lipids in solution, as an emulsion, or as a dried extract.

Certain embodiments of the present invention which include at least one method of embodiments 1a-1d represent new innovation in important aspects and/or purposes of sample extraction. For example, and without limiting the disclosure: (a) at least one embodiment is directed to extracting lipids that are not LAs or LTAs and/or have molecular weights less than 1000 m/z from a microbial sample, for the purpose of identifying in the microbial sample via mass spectrometry a taxon or non-taxon group of at least one organism at the genus level or higher, such as without limitation genus or Gram-stain status; (b) at least one embodiment is directed to extracting, at a temperature that is moderately elevated or not elevated, lipids that are not LAs or LTAs and/or have molecular weights less than 1000 m/z from a microbial sample, for the purpose of identifying in the microbial sample via mass spectrometry a taxon or non-taxon group of at least one organism at the genus level or higher, such as without limitation genus or Gram-stain status; (c) at least one embodiment is directed to extracting lipids that are not LAs or LTAs and/or have molecular weights less than 1000 m/z from a microbial sample, for the purpose of determining and/or measuring the antimicrobial resistance status of at least one organism in the microbial sample; (d) at least one embodiment is directed to extracting, at a temperature that is moderately elevated or not elevated, lipids that are not LAs or LTAs and/or have molecular weights less than 1000 m/z from a microbial sample, for the purpose of determining and/or measuring the antimicrobial resistance status of at least one organism in the microbial sample; (e) at least one embodiment is directed to extracting, at a temperature that is moderately elevated or not elevated, a mixture of deuterated and undeuterated lipids from a microbial sample or a non-microbial cell-culture sample; (f) at least one embodiment is directed to extracting a mixture of deuterated and undeuterated lipids that are not LAs or LTAs and/or have molecular weights less than 1000 m/z from a microbial sample or a non-microbial cell-culture sample.

### Embodiment 2: Mass Spectrometric Analysis

1. Prepare at least one sample according to any combination of the steps of embodiments D, E, or 1a-1c, any other method herein for extracting lipids, or any other method herein appropriate for preparing a sample for mass spectrometric analysis.

2. Optionally, if step 1 produces at least one spot on a plate, apply a composition containing a MALDI matrix to at least one spot on which samples have been placed. Optionally allow the plate to dry.

3. Place at least one MALDI plate in a mass spectrometer or otherwise present some or all of the extracted lipids and/or other analytes to a mass spectrometer, then collect mass spectra from said lipids and/or other analytes. In at least one embodiment, a single spectrum is collected. In at least one embodiment, multiple spectra are collected. In at least one embodiment, multiple spectra with different m/z (mass to charge ratio) ranges are collected. In at least one embodiment, at least one spectrum is collected with a lowest m/z of less than 300 m/z or about 300, 600, 660, 700, 800, or 1000 m/z. In at least one embodiment a spectrum is collected with a lowest m/z of greater than 1000 m/z. In at least one embodiment a spectrum is collected with a highest m/z of less than 400 m/z or about 400, 500, 600, 700, 780, 800, 1000, 1200, 1500, 1800, 2000, 2200, 2400, or 2500 m/z. In at least one embodiment a spectrum is collected with a highest m/z of higher than 2500 m/z. In at least one embodiment, two or more spectra are collected that differ in mass spectrometer parameters other than mass range, such as without limiting the invention in any way, detector gain. In at least one embodiment, at least one mass spectrum is collected that is a MALDI mass spectrum. In at least one embodiment, at least two mass spectra are collected that are MALDI mass spectra, collected from the same spot on a MALDI plate. In at least one embodiment, at least two mass spectra are collected that are MALDI mass spectra, collected from different spots on the same or different MALDI plates.

In at least one embodiment, a mass spectrometer is used that is an electrospray mass spectrometer, a desorption electrospray ionization (“DESI”) mass spectrometer, a time-of-flight mass spectrometer, a quadrupole mass spectrometer, a triple quadrupole mass spectrometer, a magnetic sector mass spectrometer, an ion trap mass spectrometer, a quadrupole trap mass spectrometer, an orbitrap mass spectrometer, a gas chromatograph mass spectrometer, a matrix-assisted laser desorption/ionization (“MALDI”) mass spectrometer, a Time-of-Flight Secondary Ion Mass Spectrometry (“TOF-SIMS”) mass spectrometer, an ion mobility mass spectrometer, a plasma chromatograph, an inductively-coupled plasma mass spectrometer, a mass cytometer, an accelerator mass spectrometer, a Fourier transform mass spectrometer, a Fourier-transform ion cyclotron resonance mass spectrometer, a mass spectrometer using an ambient ionization method such as direct analysis in real time, a mass spectrometer using a nebulization-ionization method such as surface acoustic wave nebulization, a mass spectrometer using Rapid Evaporative Ionization Mass Spectrometry, or another type of mass spectrometer.

In at least one embodiment at least one spectrum is collected that is a precursor ion spectrum, a tandem spectrum, an MSⁿ spectrum, or a spectrum of a different type. In at least one embodiment, spectra are produced via data-dependent precursor ion selection. In at least one embodiment, spectra are produced via multiple reaction monitoring (“MRM”), a selected reaction monitoring (“SRM”) other than MRM such as without limitation consecutive reaction monitoring (“CRM”) or parallel reaction monitoring (“PRM”), a data-dependent precursor ion selection technique other than SRM, or a data-independent precursor ion selection technique, such as without limiting the invention in any way, Precursor Acquisition Independent From Ion Count (“PAcIFIC”) (Panchaud 2099 doi: 10.1021/ac900888s).

### Embodiment 3: Spectroscopic Analysis

1. Prepare at least one sample according to any combination of the steps of embodiments D, E, or 1a-1c, any other method herein for extracting lipids, or any other method herein appropriate for preparing a sample for spectroscopic analysis.

2. Analyze the at least one sample with a spectroscopic instrument, where the aforementioned spectroscopic instrument operates by laser induced fluorescence spectroscopy, atomic absorption spectroscopy, atomic emission spectroscopy, flame emission spectroscopy, acoustic resonance spectroscopy, cavity ring down spectroscopy, circular dichroism spectroscopy, Raman spectroscopy, surface enhanced Raman spectroscopy, coherent Raman spectroscopy, cold vapor atomic fluorescence spectroscopy, nuclear magnetic resonance spectroscopy, electrical impedance spectroscopy, electron phenomenological spectroscopy, electron paramagnetic resonance spectroscopy, Fourier-transform spectroscopy, laser-induced breakdown spectroscopy, photoacoustic spectroscopy, photoemission spectroscopy, photothermal spectroscopy, spectrophotometry, vibrational circular dichroism spectroscopy, gamma spectroscopy, flow cytometry, or some other type of spectroscopy; or by means of a scintillation detector, scintillation counter, Geiger counter, ionization chamber, gaseous ionization detector, or other radiation detector.

### Embodiment 4: Combined Analysis

Collect both mass spectrometric and spectroscopic information on at least one sample, by performing at least one of the following steps 1-3.

1. Perform the steps of both embodiment 1 and embodiment 3. In at least one embodiment, embodiment 2 is performed before embodiment 3, embodiment 3 is performed before embodiment 2, embodiments 2 and 3 are performed in parallel, or embodiments 2 and 3 can be performed in any order or sequence. In at least one embodiment, embodiment 2 and embodiment 3 are performed on the same sample or sample container. In at least one embodiment, at least two different samples or container are used for performing at least one of embodiment 2 or embodiment 3. In at least one embodiment, embodiment 2 and embodiment 3 are performed on one or more spots on the same MALDI plate or other sample plate. In at least one embodiment, at least two spots on the same or on different MALDI or other sample plates are used for performing at least one of embodiment 2 or embodiment 3.

2. Perform the steps of embodiment 2, then using the same plate perform the steps of embodiment 3 beginning with step 2 of embodiment 3.

3. Perform the steps of embodiment 3, then using the same plate perform the steps of embodiment 2, beginning with step 2 of embodiment 2.

### Embodiment 5a: Classification of Spectra

1. Obtain at least one spectrum using any method of embodiments 2-4 or 21, or any other method herein.

2. Optionally preprocess at least one spectrum obtained in step 1. For example, and without limiting the disclosure, in at least one embodiment, at least one spectrum is preprocessed by at least one of baseline correction, mass alignment, charge state deconvolution, isotopic deconvolution, Fourier transform, a type of integral transform other than Fourier transform, peak extraction, or some other kind of feature extraction. In at least one embodiment, a sequence of preprocessing operations is performed on one or more spectra and at least one additional sequence of preprocessing operations is performed on the one or more spectra and/or on one or more additional spectra, where each the aforementioned spectra is obtained from a distinct sample or alternatively at least two of the aforementioned spectra are obtained from the same sample.

3. Using at least one spectrum obtained in step 1, optionally preprocessed in step 2, perform classification using a classification method.

For example, and without limiting the disclosure, in at least one embodiment at least one spectrum is classified by comparing the spectrum to an exemplar, average, consensus, or synthetically generated spectrum. As a further example, in at least one embodiment, at least one spectrum is classified partly or entirely without comparison to another spectrum. For example, and without limiting the disclosure, in at least one embodiment at least one spectrum is classified using a machine learning method such as without limitation one or more of support vector machines, neural networks, non-negative alternating least squares (“NNLS”), constraint optimization, linear programming, ensemble learning, decision trees, decision forests, random forests, gradient boosting, regression analysis, feature selection, and/or feature learning. One skilled in the art will appreciate that certain machine learning methods are typically performed using machine learning models that have previously been trained on training data with properties related to the data to be classified.

In at least one embodiment, classification is performed on a single spectrum or on features extracted from a single spectrum from at least one sample. In at least one embodiment, classification is performed on at least two spectra or on features extracted from at least two spectra from at least one sample. In at least one embodiment, the aforementioned at least two spectra are from a single spot on a MALDI plate. In at least one embodiment, a first spectrum of at least two spectra is from a first spot on a first plate whereas at least one other spectrum of the at least two spectra is from a different spot on the first plate or on a second plate. In at least one embodiment, three or more spectra from one or more spots are used. In at least one embodiment, at least two spots are processed under essentially the same conditions. In at least one embodiment at least two spots are processed differently.

In at least one embodiment, at least one spectrum used for training data and/or at least one exemplar, average, or consensus spectrum is produced by a method described in one of the embodiments or elsewhere herein. In at least one embodiment, at least one spectrum used for training data and/or at least one exemplar, average, or consensus spectra is produced by some other method besides a method described in one of the embodiments or elsewhere herein.

### Embodiment 5b: Quantitative Estimates from Spectra

1. Obtain at least one spectrum using any method of embodiments 2-4, or 21, or any other method herein.

2. Optionally preprocess at least one spectrum obtained in step 1. For example, and without limiting the disclosure, in at least one embodiment, at least one spectrum is preprocessed by at least one of baseline correction, mass alignment, charge state deconvolution, isotopic deconvolution, Fourier transform, a type of integral transform other than Fourier transform, peak extraction, or some other kind of feature extraction. In at least one embodiment, a sequence of preprocessing operations is performed on one or more spectra and at least one additional sequence of preprocessing operations is performed on the one or more spectra and/or on one or more additional spectra, where each the aforementioned spectra is obtained from a distinct sample or alternatively at least two of the aforementioned spectra are obtained from the same sample.

3. Using at least one spectrum obtained in step 1, optionally preprocessed in step 2, estimate a quantity using an estimation method.

For example, and without limiting the disclosure, in at least one embodiment at least one quantity is estimated for at least one spectrum using a method from embodiment 5a. As a further example without limiting the invention in any way, in at least one embodiment, NNLS is used to estimate a quantity for at least one spectrum. As a further example without limiting the invention in any way, in at least one embodiment, NNLS is used to estimate the ratio of two chemical species in a mass spectrum by comparison of vectors consisting of peak intensities corresponding to the isotopic distributions of said two chemical species, said isotopic distributions primarily caused by the ratio of ¹³C to ¹²C atoms in said two chemical species. As a further example without limiting the invention in any way, in at least one embodiment, NNLS is used to estimate in a sample the ratio between at least one ion of a deuterated lipid and at least one ion of the same or a different undeuterated lipid.

In FIG. 3 is shown a MALDI mass spectrum from about 700 to 760 m/z in negative ionization mode of E. coli prepared via the methods of embodiments 1b and 2.

It will be appreciated by one skilled in the art that MALDI mass spectrometry predominately produces singly-charged ions, so that m/z values are mostly and most often mass values, giving consideration to possible mass loss or gain during ionization. Herein, in reference to MALDI mass spectrometry, “mass” and “m/z” should be interpreted as synonymous unless from context the sense is otherwise.

In FIG. 4 is shown synthetic parent ion mass spectra for four PG lipid species and one PE lipid species as shown. The synthetic spectra account for [M−H]-ionization in a mass spectrometer. The shape of each peak is produced by a simple mathematical estimate of a mass spectrometer ion peak, but in at least one embodiment, a more complex estimate of mass spectrometer peak shape of a type familiar to one skilled in the art is used. By the method well known to those skilled in the art, the synthetic spectra further account for predicted isotopic distributions as a result of common naturally-occurring isotopes, most importantly the ¹²C-¹³C ratio and the number of carbon atoms in each lipid. The relative intensity of ion peaks for each lipid was weighted via NNLS to best fit the typical pattern of E. coli as seen for example in FIG. 3. One skilled in the art will appreciate that for each exemplar lipid molecular species shown, there are additional molecular species with a mass indistinguishable from the examplar molecular species using common mass spectroscopy instruments and differing in each case from the exemplar lipids for example by the relative length of side chains and/or the position of one or more double bonds on side chains; said additional species typically produce parent ion spectra that are indistinguishable from the example species on mass spectrometers of typical resolution, and in at least one embodiment a comparison is made to ion peaks as described in this embodiment and elsewhere herein without determining if any particular peak in at least one mass spectrum is produced by any particular such species or by more than one such species.

In FIG. 5 is shown synthetic spectra for eight PE lipid species and four PG lipid species, using the same approach as for FIG. 3. Inverted under said spectra is an integrated sum spectrum.

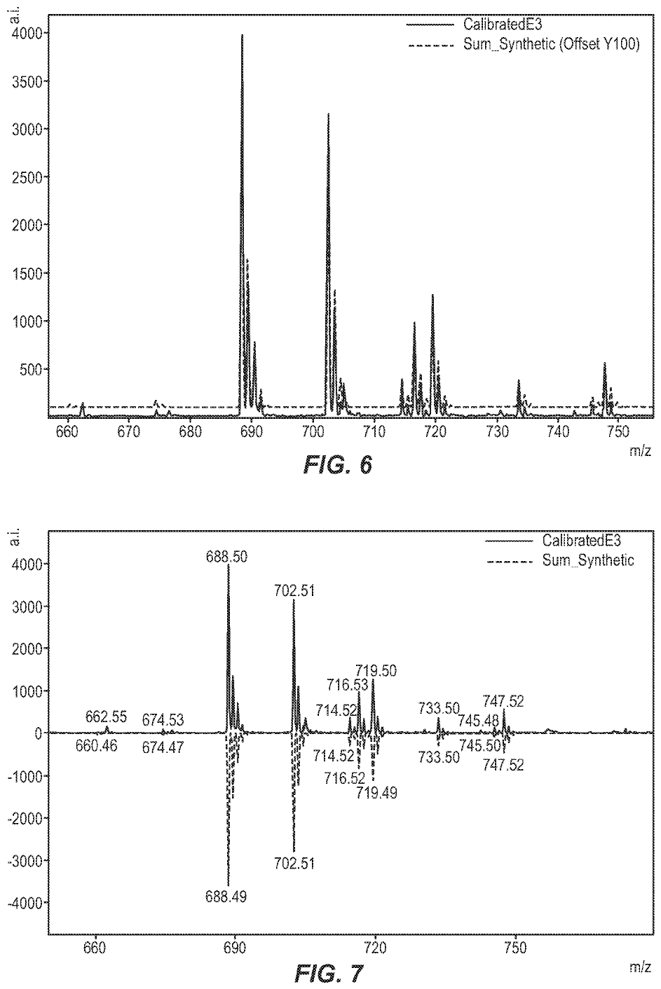

In FIG. 6 is shown the synthetic sum spectrum from FIG. 4 superimposed on the E. coli spectrum from FIG. 3. The spectra in FIG. 6 are also shown in FIG. 7, with the synthetic spectrum inverted and ion peaks labeled as follows: apparent monoisotopic peaks are labeled in the E. coli spectrum and calculated monoisotopic peaks are labeled in the sum spectrum.

One skilled in the art will appreciate that FIGS. 3-7 demonstrate that PE and PG lipids explain the peaks shown in E. coli spectra in the mass range of about 700 to 760 m/z. One skilled in the art will further appreciate that subject to differences in ionization efficiencies which may not be known and do not need to be known, the relative abundance of PE-PG lipids in a sample including a biological sample can be modeled and predicted via techniques including without limitation NNLS.

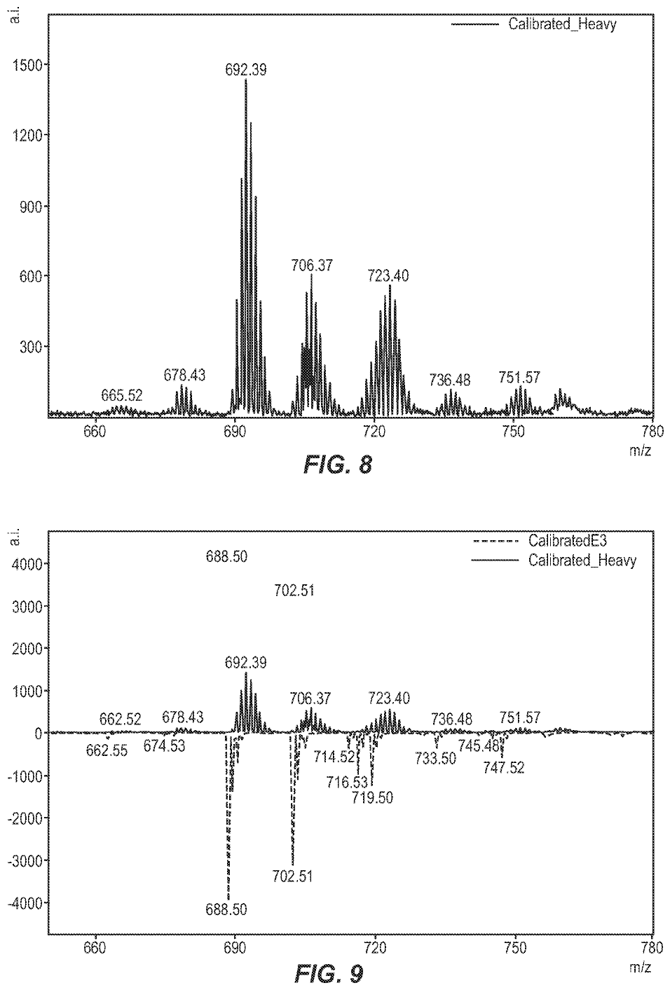

In FIG. 8 is shown a MALDI mass spectrum from about 660 to 780 m/z in negative ionization mode of the same strain of E. coli as in FIG. 3 and prepared via the same methods as FIG. 3 except that the environment used to grow the bacteria contained about 8% D₂O water by molarity. In FIG. 9, the spectra of FIGS. 3 and 8 are compared, wherein it can be observed that the addition of deuterium causes an isotopic distribution from D addition to be superimposed on the existing predominantly ¹²C-¹³C isotopic distribution.

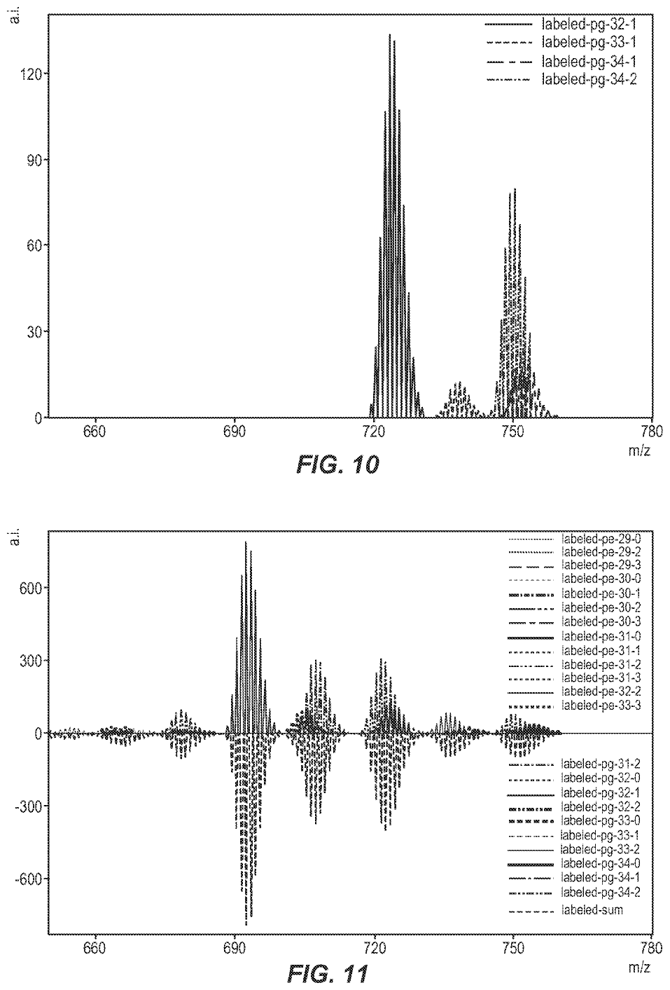

In FIG. 10 is shown a synthetic parent ion mass spectrum for the four PG lipid species masses as shown in FIG. 4, accounting for [M−H]-ionization in a mass spectrometer, a predicted ¹³C-¹²C isotopic peak distribution based on the number of carbon atoms in each lipid, and a predicted H-D isotopic distribution based on 8% deuterium concentration at equilibrium. The intensity of each group of peaks was weighted via NNLS to best fit the pattern of E. coli exemplified in FIG. 8.

In FIG. 11 is shown synthetic spectra for 13 PE lipid species and 10 PG lipid species, using the same approach as for FIG. 10. Inverted under said spectra is an integrated sum spectrum.

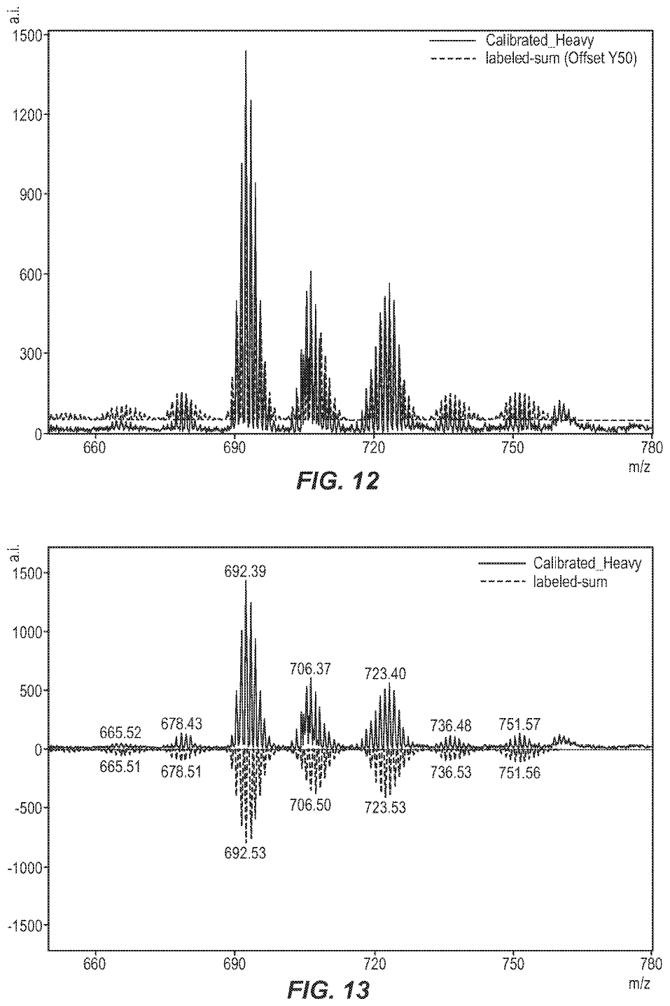

In FIG. 12 is shown the synthetic sum spectrum from FIG. 11 superimposed on the E. coli spectrum from FIG. 8. The spectra in FIG. 12 are also shown in FIG. 13, with the synthetic spectrum inverted and ion peaks labeled as follows: apparent monoisotopic peaks are labeled in the E. coli spectrum and calculated monoisotopic peaks are labeled in the sum spectrum.

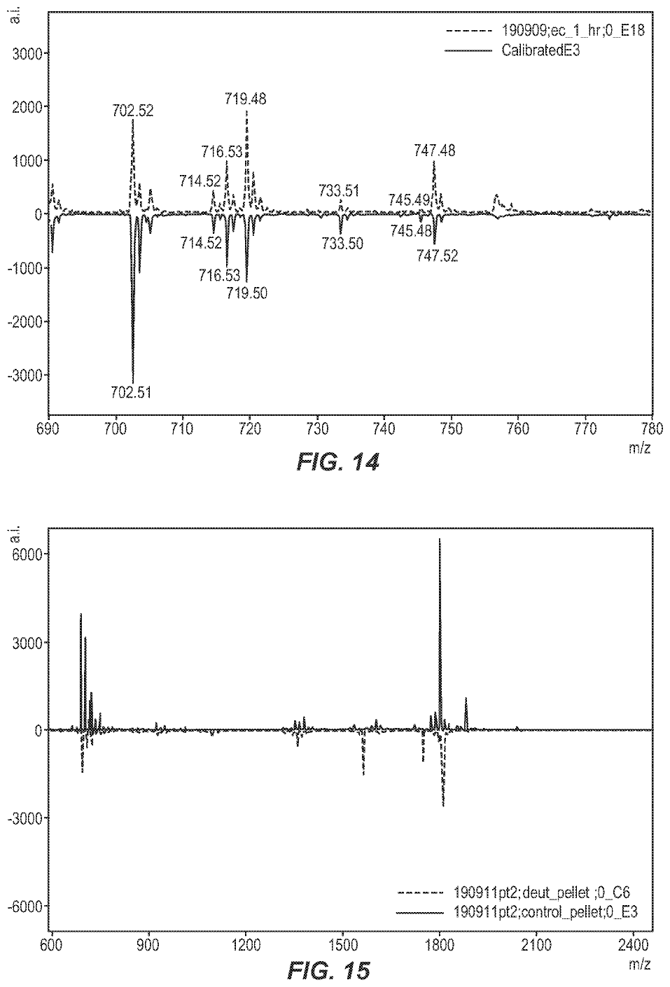

One skilled in the art will appreciate that FIGS. 8-13 demonstrate that deuteration further increases confidence that PE and PG lipids produce the peaks shown in E. coli spectra in the mass range of about 660 to 760 m/z, One skilled in the art will further appreciate that if the H-D ratio of a sample is known, the ratio of undeuterated to deuterated molecular species in a sample including a biological sample can be modeled and predicted via techniques including without limitation NNLS. In FIG. 14 are shown two mass spectra. The upper spectrum is from a sample prepared as in FIG. 8, except that the sample was grown in broth with 20% enriched deuterium. To retard growth the broth was placed in a narrow tube and the tube was not agitated. The lower spectrum is the same spectrum as in FIG. 3. As can be easily seen and as will be readily appreciated by one skilled in the art, the two spectra in FIG. 14 are highly similar, and there is no evidence of deuterated ion peaks. Thus, deuteration of classes of cellular molecules including phospholipids can be used to detect cellular growth, the absence of deuterated peaks indicates the absence of growth below a limit of detection, and in an environment containing a known H-D ratio the ratio of the amount of new growth in said environment to the amount of cells originally placed in said environment can be estimated.

In at least one embodiment, a sample is grown in an environment containing a known H-D ratio and microbial or other cellular growth in said environment and/or measure and/or estimate the degree or amount of growth in said environment. In at least one embodiment, an H-D ratio of at least 10:1 is used, resulting in good signal-to-noise ratio of the aforementioned measurements. In at least one embodiment, an environment containing a known H-D ratio and an antimicrobial agent is used as an antimicrobial susceptibility test (“AST”). In at least one embodiment, an environment containing a known H-D ratio and optionally containing one or more materials eliciting one or more environmental responses from one or more species or non-species classifications of organisms is used to evaluate and/or classify a sample containing one or more species and/or non-species classifications of organisms.

### Embodiment 5c: Combined Analysis from Spectra

Perform at least one of steps 1-3 using at least one spectrum obtained by any appropriate method herein.

1. (a) Using a method of embodiment 5a, make a classification; (b) using a method of embodiment 5b, estimate a quantity. The same spectrum or spectra or different spectra may be used in (a) and (b). For example, and without limiting the disclosure, (a) using a MALDI mass spectrum from about 650 to 850 m/z, classify an undeuterated spectrum as plausibly produced by a Gram-positive organism, a Gram-negative organism or fungus, both of the first two classes, or neither; then (b) use a quantitative estimating model specific to the class identified in (a) to estimate growth in a second, deuterated spectrum.

2. (a) Using a method of embodiment 5b, estimate a quantity; (b) using a method of embodiment 5a, make a classification. For example, and without limiting the disclosure, from a sample grown in a deuterated environment with an antimicrobial agent, obtain a first mass spectrum from about 650 to about 850 m/z and a second mass spectrum from about 1000 to about 2400 m/z, then (a) using a method of embodiment 5b, estimate the relative fraction of growth in the deuterated environment in the sample producing the spectra; (b) use the growth estimate as one of at least one variables for a classification model for whether the organism in the aforementioned sample expresses or does not express an antimicrobial phenotype.

3. Combine the methods of embodiments 5a and 5b in a more complex pattern. For example, and without limiting the disclosure, obtain MALDI mass spectra of a bacterial specimen grown in different concentrations of an antibiotic in deuterium-enriched environments. Use a method of 5a to estimate the degree of growth shown in each spectrum, then use a method of 5b and the growth estimates to classify the sample as having sufficiently exited lag phase or not having sufficiently exited lag phase. If the sample has sufficiently exited lag phase, use a method of embodiment 5b to estimate the degree of growth in each spectrum. From the aforementioned degrees of growth in each spectrum, using any method of embodiment 5b or a standard method for linear regression, interpolate or extrapolate the growth vs. antibiotic concentration curve for the sample, and from said curve estimate the MIC for the bacterial sample and the antibiotic.

### Embodiment 6: Classification of Samples

1. Obtain at least one spectrum from at least one sample by performing steps from embodiments 1-4 or 21 or any other method herein.

2. Classify samples by performing steps of at least one of embodiments 5a-5c one or more times on the at least one spectrum obtained in step 1.

In at least one embodiment, at least one sample is classified hierarchically. The at least one sample is first classified at a more general level by performing steps of one or more of embodiment 5a-5c for a first classification, then the at least one sample is classified to a more specific level by performing steps of one or more of embodiment 5a-5c again for a second classification; wherein either the same spectrum or spectra are used for the aforementioned first classification and second classification or else at least one spectrum is used for the first classification and not the second classification, and/or at least one spectrum is used for the second classification and not the first classification. Furthermore, in at least one embodiment, the aforementioned hierarchical classification is extended in like manner to a third classification step or to any number of classification steps.

### Embodiment 7: Screen Samples

1. According to the steps of embodiment 6, using at least one spectrum, classify at least one sample for the presence of microbes or for the presence of microbes above a threshold amount, or conversely classify at least one sample for the absence of microbes or for the absence of microbes below a threshold amount, or classify at least one sample for the presence or absence or for the presence or absence above or below a threshold amount of a taxon of microbe such as without limitation bacteria, or for the presence or absence or for the presence or absence above or below a threshold amount of one or more categories not corresponding to taxa, such as without limitation Gram stain. In a preferred embodiment, at least one spectrum is a Raman spectrum.

2. Optionally, If in step 1 the aforementioned sample is determined to have microbes of interest present or present above a threshold value, then according to the steps of embodiment 6, using the same or different at least one spectrum as in step 1, classify the sample according to at least one of microbial species, strain, taxon above the level of species, antimicrobial susceptibility or resistance, virulence, and/or one or more other categories.

For example, and without limiting the disclosure, in a preferred embodiment, a precursor mass spectrum of a sample determines the presence or absence of microbes belonging to one or more classes in the sample, said one or more classes either corresponding to taxa or not corresponding to taxa, then one or more tandem mass spectra determine the identity of microbes present at a greater refinement than was determined with the precursor spectrum, such as without limitation microbial species, strain, taxon above the level of species, antimicrobial susceptibility or resistance, or virulence.

### Embodiment 8: Classify Samples Via Mass Spectrometry

1. According to the steps of embodiment 6, using at least one mass spectrum, classify a sample according to at least one of microbial species, strain, taxon above the level of species, strain, antimicrobial susceptibility or resistance, virulence, and/or one or more other categories.

### Embodiment 9: Classify Samples Via Electrospray Mass Spectrometry

1. According to the steps of embodiment 6, using at least one electrospray mass spectrum, classify a sample according to at least one of microbial species, strain, taxon above the level of species, strain, antimicrobial susceptibility or resistance, virulence, and/or one or more other categories.

### Embodiment 10: Classify Samples Via Spectroscopy

1. According to the steps of embodiment 6, using at least one spectrographic spectrum, classify a sample according to at least one of microbial species, strain, taxon above the level of species, strain, antimicrobial susceptibility or resistance, virulence, and/or one or more other categories.

### Embodiment 11: Kits

In at least one embodiment, at least one kit is used, said kit consisting in part or whole of one or more instances of at least one of the following: a deuterated solution, microbial growth medium, a cell culture medium that is not or is not necessarily limited to culture of microbes, an antimicrobial-containing solution, a solution containing a growth modification and/or inhibition agent that is not or is not necessary an antimicrobial agent, an antimicrobial susceptibility solution, a microtiter plate or similar structure to support culture of microorganisms in one or more chambers or wells, an antimicrobial susceptibility test, a blood culture bottle or chamber, a MALDI plate that optionally has additional functions including without limitation performing lipid extraction and/or performing microbial cell culture, a MALDI matrix solution, a material that when dissolved in a liquid can be applied to form a MALDI matrix, a redox indicator or other pH indicator, whether colorimetric or otherwise, a reservoir, a needle, a buffer solution, an acidic solution, a solid to which a liquid can be added to form a buffer or acidic solution, a matrix solution, a device for heating plates or needles or both or similar or other small objects, instructions, and a software program and/or license key and/or other rights or credentials to a software program or software service that with or without additional data classifies samples and/or spectra according to any of the embodiments and/or any of the methods described herein.

In at least one embodiment, a kit contains at least one object not mentioned in the preceding description. In at least one embodiment, a kit contains more than one instance of at least one object mentioned in the preceding description.

### Embodiment 12: Membrane Lipids

In FIG. 28 is shown example lipid types for three classes of microbes: Gram-positive bacteria, Gram-negative bacteria, and fungi. In FIG. 4 is shown exemplar phospholipids found in the membranes of many species of bacteria. In at least one embodiment, for at least one taxon, species, strain, clone or other classification of microbe, at least one type of said example lipid types is extracted. In at least one embodiment, for said classes of microbes, for at least one of said class of microbe, at least one lipid is extracted that is not shown in FIG. 28.

### Embodiment 13: Lipid A

In FIG. 29 is shown an example structure of lipid A. This structure is associated in the literature with lipid A from E. coli, observed at a nominal mass of about 1798 m/z.

### Embodiment 14: Determining Ions Derived from Metabolic Pathways

In FIGS. 15-19 is shown different m/z ranges of two spectra. In each figure, the upper spectrum is from E. coli grown in an undeuterated (containing no added deuterium above natural abundance) environment and the lower spectrum is from the same strain of E. coli grown in an deuterated (containing a deuterium level above the natural abundance) environment containing water that is about 8% D₂O by molarity (“8% deuterated environment.”) Both E. coli samples were grown according to the methods of embodiments 1a and 2. The spectra shown are not calibrated, resulting in high m/z values being inaccurate by several m/z.

FIG. 15 shows the complete spectra from 600 to 2500 m/z.

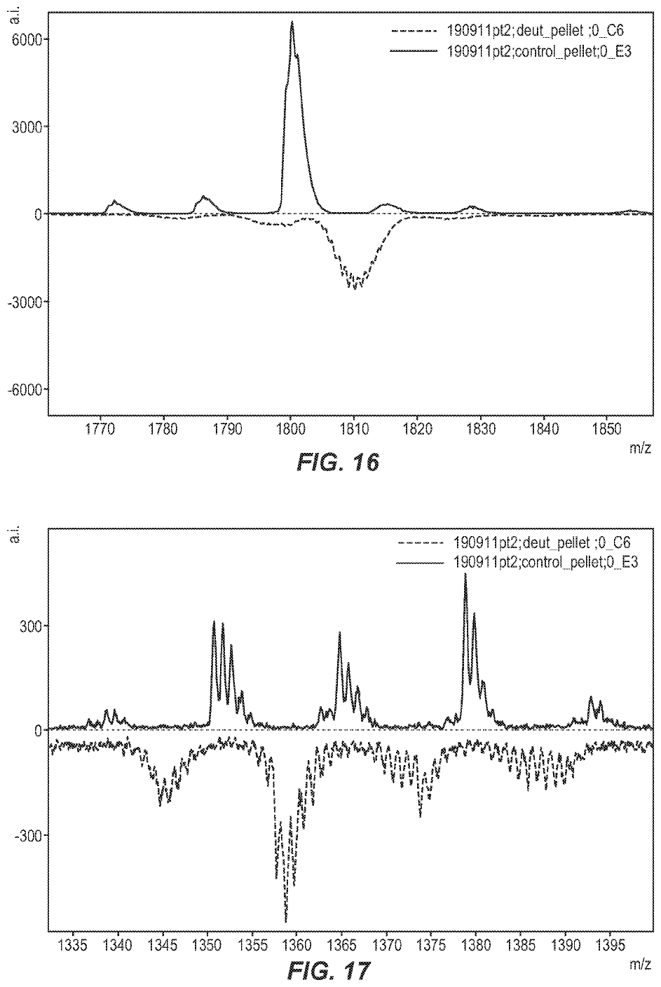

FIG. 16 shows a region of the spectra near 1800 m/z. The base peak ion at about 1798 m/z in the upper spectrum is characteristic for E. coli, and corresponds to the structure shown in FIG. 29. The peaks around 1810 m/z in the lower spectrum are deuterated ions corresponding to the aforementioned ions in the upper spectrum. Thus, lipid A was substantially produced only during the growth phase of E. coli. Further, lipid A is produced on a pathway where its fatty acid chains are exposed to H-D exchange. In fact it is reported (Zhang 2017 DOI: 10.1021/jacs.7b08012) that the fatty acid chains of lipid A are produced from NADPH, and in this process, hydrogens ultimately bound to the carbons that will later comprise the aforementioned fatty acid chains are exposed to H-D exchange. As previously mentioned, hydrogens bound to straight-chain carbons do not ordinarily experience H-D exchange, so the lower spectrum contains almost no ions produced before E. coli began growing in the deuterated environment, but does contain ions produced after the E. coli began growing in the deuterated environment.

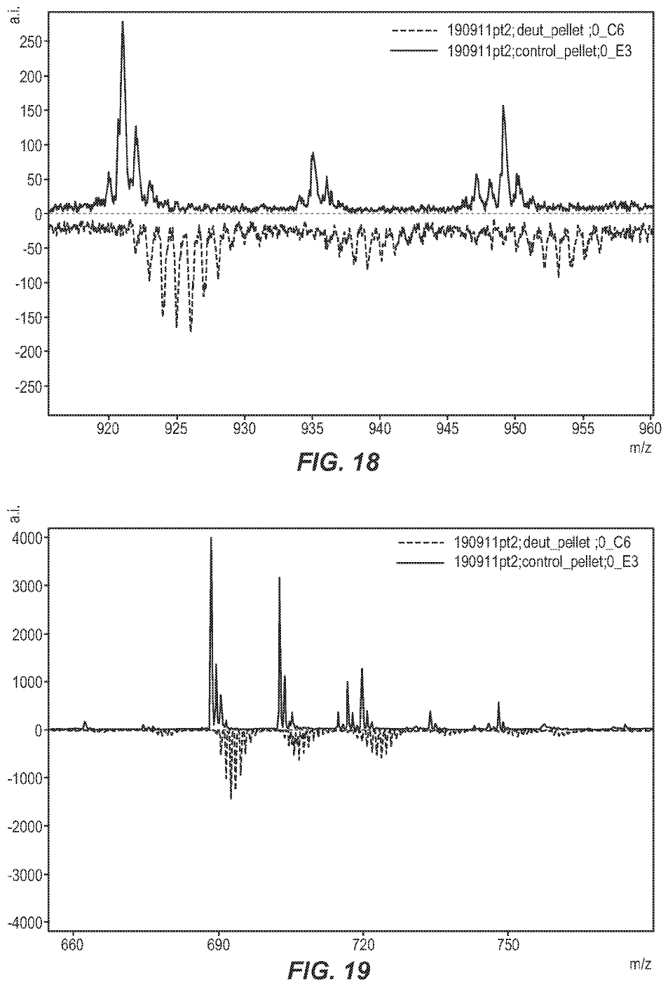

FIGS. 17 and 18 show m/z ranges as indicated. FIGS. 17 and 18 each show evidence of three chemical species produced by a deuterated pathway during growth phase.

FIG. 19 shows the m/z range from about 660 to about 780 m/z, which has also been discussed above. Like the region around 1800 m/z, this region has strong signal intensity, and consequently a low LOD.

From FIGS. 15-19 it can be observed that E. coli produces multiple chemical species via one or more H-D exchanging pathways.

### Embodiment 15: Determining Growth

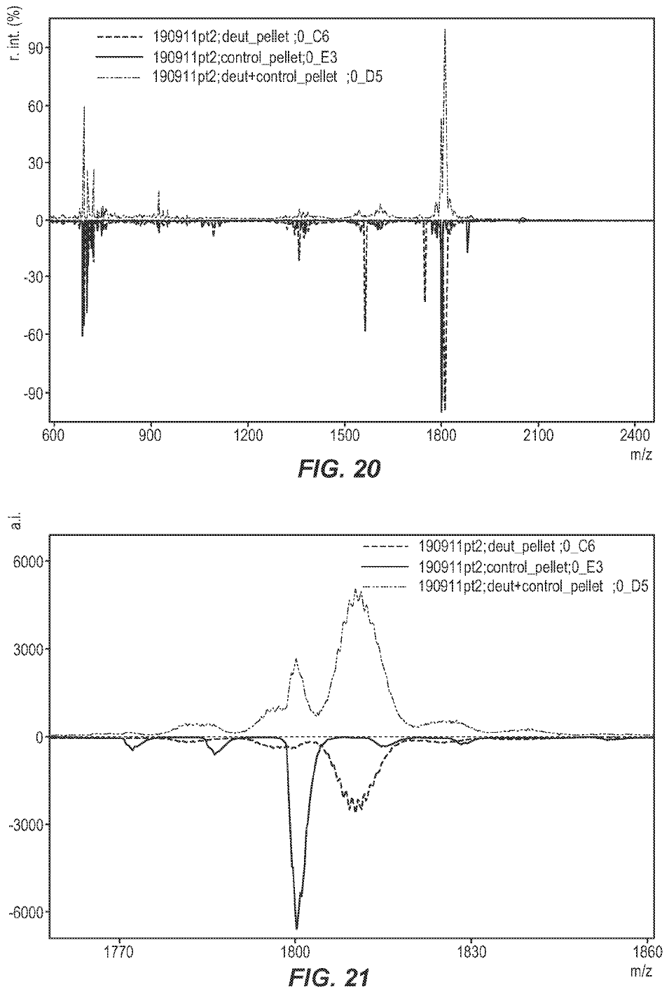

In FIGS. 20-24 are shown MALDI mass spectra for three samples, for different m/z ranges in each figure, each figure showing one spectrum for each sample. The samples are an undeuterated E. coli sample, an E. coli sample grown in an 8% deuterated environment, and a sample made by combining the deuterated and undeuterated E. coli samples. All three samples were prepared by the methods of embodiments 1a and 2. The spectra shown are not calibrated, resulting in high m/z values being inaccurate by several m/z.

FIG. 20 shows the complete spectra from 600 to 2500 m/z.

FIG. 21 shows the m/z region around 1800 m/z, similar to FIG. 16. It can be observed that the combined spectrum contains the significant ion peaks from both the deuterated and undeuterated spectra.

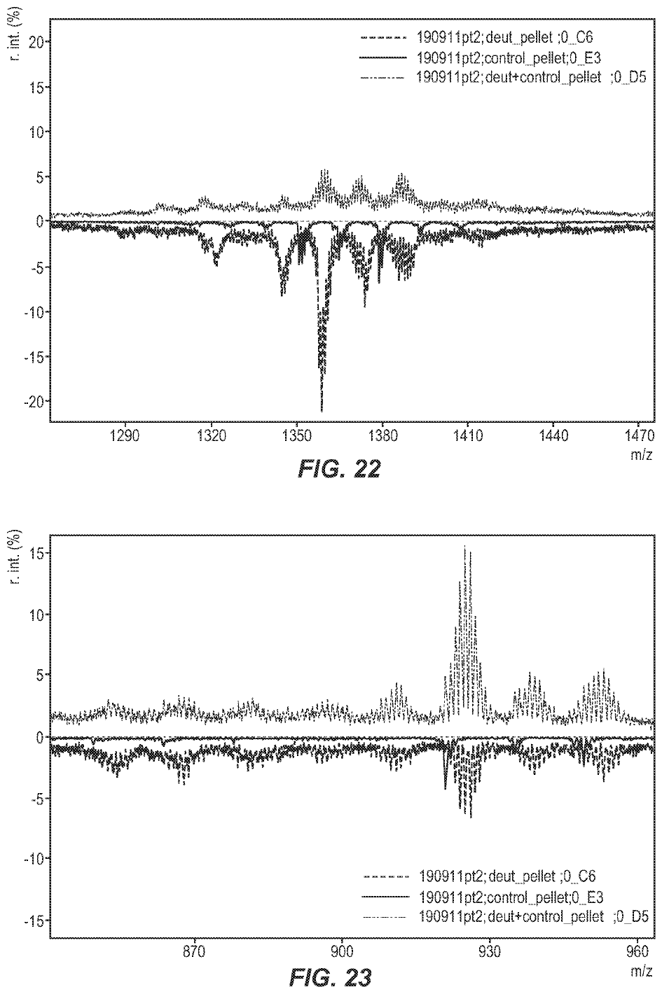

FIGS. 22 and 23 show additional m/z ranges of the spectra. These regions have low signal intensity and lower signal/noise. Nonetheless, significant peaks from both the deuterated and undeuterated spectra can be observed in the combined spectrum.

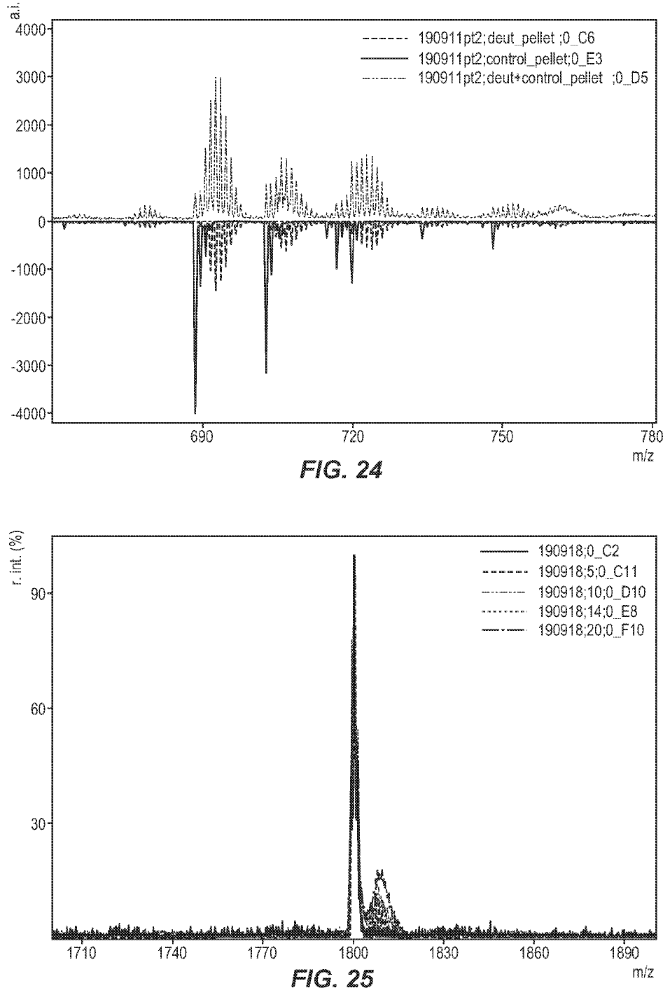

FIG. 24 shows the m/z range of about 660 to 780 m/z. As with FIG. 20, the combined spectrum contains the significant ion peaks from both the deuterated and undeuterated spectra.

From FIGS. 20-24 it can be observed that a mixture of samples of a species of bacteria grown in deuterated and undeuterated environments produces lipid membrane MS ions that correspond with a mixture of the mass spectrometry signals produced by the same bacterial species grown in deuterated and undeuterated environments and analyzed separately.

In FIG. 25 can be seen mass spectra from cultures of E. coli grown in 8% deuterated environment for progressively longer times at 60 min. intervals, from 0 min. to 240 min. A 1 μL sample was then extracted from each sample using the method of embodiment 1a and MALDI mass spectra collected using the method of embodiment 2 using negative ionization mode. The mass spectra are normalized to the intensity of the peak at about 1800 m/z. corresponding in this uncalibrated spectrum to the characteristic LA ion of E. coli. As can be seen from FIG. 25, the intensity of the ion peaks to the right of the peak at about 1800 m/z progressively increases as growth time increases. As has been demonstrated in previous embodiments, this second group of peaks increases in intensity progressively with longer growth times. Thus, as E. coli grows in a deuterated environment, the ratio of deuterated to undeuterated lipid peaks progressively increases.

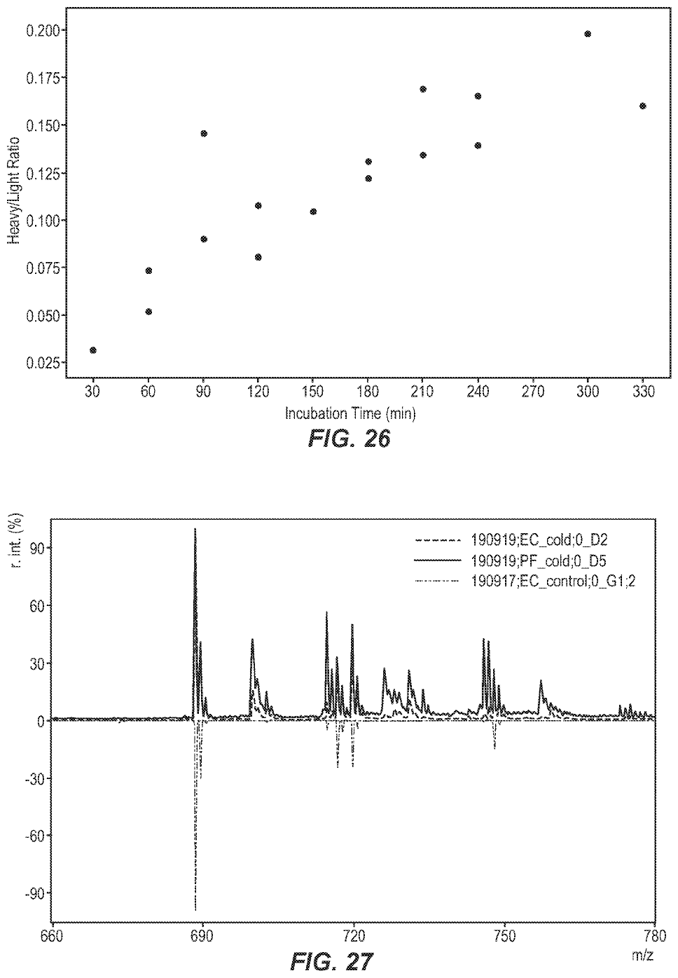

In FIG. 26 can be seen a graph of the ratio of heavy/light LA ions from the spectra in FIG. 25 and additional spectra that were not shown in FIG. 25 for clarity, where light LA is measured as the intensity of the ion peak at 1797 m/z. and heavy LA is measured as the intensity of the ion peak at 1808 m/z. As can be seen from FIG. 26, an approximately linear relationship between growth time and the ratio of these two ion intensities is a satisfactory explanation for the data in FIG. 26. Furthermore, as can be seen from FIGS. 25 and 26, growth time is trivially estimated from deuterated mass spectra, using even the simplest of analytical techniques. Furthermore, growth can be observed at 30 min. and the amount of growth at 60 min. can be distinguished from the amount of growth at 30 min.

One skilled in the art will appreciate that in the foregoing example, instead of using single ion peaks at for example 1808 m/z, that multiple peaks could have be used, to improve the sensitivity and/or accuracy of the measurement.

By analogy with a “transition” in Multiple Reaction Monitoring, a “deuteration,” or “deuterium transition” herein means one or more expected deuterated peaks, given an undeuterated peak and the assumption or knowledge that the peak is produced by a molecule with a certain number of hydrogens that do not exchange with solution after synthesis.

One skilled in the art will appreciate that in the foregoing example, instead of or in addition to using a single deuterium transition at 1797, deuterium transitions for example as shown in FIG. 24 could have been used. Having multiple choices for deuterium transitions increases the chance that a suitable measurement can be made from a given sample. Using multiple deuterium transitions allows for the averaging or other combination of multiple measurements for greater accuracy. Certain ions have specific advantages, sometimes depending on the microbial species in the sample. For example, and without limiting the disclosure, the ion peaks shown in FIG. 24 are common to many Gram-negative bacteria, are easier to extract than some other lipids, and are at a mass range where mass spectrometers typically have good resolution and sensitivity. As a further example without limiting the invention in any way, LA is a major component of the Gram-negative membrane, and is typically easily extracted, producing good intensity ions at different specific masses for each Gram-negative bacterial species, allowing growth of individual microbial species in a polymicrobial sample to be determined.

In at least one embodiment, a method of this embodiment 15 is used to determine growth of at least one cell, cell culture, tissue, microbial community, biofilm, or similar. In at least one embodiment a method of this embodiment 15 is used to determine growth of a sample from embodiments A-C. In at least one embodiment, a method of this embodiment 15 is used on a sample prepared according to a method of embodiments D, E or 1a-1e. In at least one embodiment, a method of this embodiment 15 is used to determine growth of a sample analyzed by a method of embodiments 2-4, 5a-5c, 6-10, 21, 22, 200-204, or 300. In at least one embodiment a method of this embodiment 15 is used to determine growth for the purpose of determining and/or measuring antimicrobial resistance and/or susceptibility. In at least one embodiment a method of this embodiment 15 is used to determine growth for the purpose of determining and/or measuring one or more of an ADME-Tox parameter or other measure, efficacy, release profile, toxicity, toxicology, and/or drug treatment effect for at least one substance.

### Embodiment 16: Cold Extraction

In FIG. 27 are shown three spectra. The upper two spectra are respectively from samples of E. coli (EC) and Pseudomonas fluorescens (PF) samples extracted using the method of embodiment 1d. The lower spectrum is from a sample of E. coli that was prepared according to a method of embodiment 1a. All three prepared samples were then analyzed via MALDI mass spectrometry according to embodiment 2 using negative ionization mode.

As can be seen from FIG. 27, a method of embodiment 1d produced spectra in the mass range 660-780 very similar to a method of embodiment 1a for E. coli, with the same ions prominent in each case. Furthermore, the method of embodiment 1d produced similar spectra in the mass range 660-780 for E. coli spectra and P. fluorescens, with most of the same ions prominent in each spectrum.

### Embodiment 17: Determining Growth Inhibition

In FIG. 1 can be seen multiple spectra for samples of a P. fluorescens strain that is susceptible to kanamycin, grown under different conditions, then extracted with a method of embodiment 1d and analyzed with a method of embodiment 2 using negative ionization mode. From bottom to top, the spectra come from samples cultured as follows according to the last letters of the legend of the figure: control: no D₂O, no kanamycin; H4: 8% D₂O, no kanamycin; G5: 8% D₂O, 0.195 μg/mL kanamycin; F5: 8% D₂O, 0.780 μg/mL kanamycin; E5: 8% D₂O, 3.125 μg/mL kanamycin; D5: 8% D₂O, 12.5 μg/mL kanamycin; C5: 8% D₂O, 50.0 μg/mL kanamycin; B5: 8% D₂O, 200 μg/mL kanamycin.

Ion m/z values are labeled on the spectra. It can be seen that certain labeled ions occur in all spectra in FIG. 1.

It can be seen that there are many labeled ions in common between the spectra in FIG. 1 and FIG. 3, showing that mass spectra from E. coli and P. fluorescens extracted by a method of embodiment 1d have many of the same MS ions in this mass range. It can be seen that there are many labeled ions in common between the spectra in FIG. 1 and FIG. 4, and between FIG. 1 and FIG. 7 showing that, similar to E. coli, the labeled ion peaks in FIG. 1 are PE and PG molecules. Additionally, the ¹²C²³C-dominated natural isotopic distribution of PE and PG molecules leads to an expected ¹³C isotopic ion approximately 1 m/z heavier than the monoisotopic peak, further accounting for ion peaks in common between the aforementioned spectra in FIGS. 1, 3, 4, and 7.

It can be seen in FIG. 1 that spectrum G16 most closely resembles spectrum B5, and likewise spectrum B5 most closely resembles spectrum G16, and that they are highly similar. Thus P. fluorescens cultured in 200 μg/mL kanamycin and 8% D₂O resembles P. fluorescens cultured in no normal water, further demonstrating that deuterated ion peaks indicate growth, P. fluorescens cultured in a high kanamycin concentration shows no evidence of growth in the form of deuterated ion peaks.

It can be further seen in FIG. 1 that spectrum G5 most closely resembles spectrum H₄, and likewise spectrum H₄ most closely resembles spectrum G5, and that they are highly similar. P. fluorescens cultured in 0.195 μg/mL kanamycin and 8% D₂O resembles P. fluorescens cultured in D₂O and no kanamycin, providing further evidence that deuterated ion peaks indicate P. fluorescens cultured in a low kanamycin concentration shows evidence of growth in the form of deuterated ion peaks.

It can be further seen in FIG. 1 that as kanamycin concentration increases, the relative intensity of deuterated ion peaks to undeuterated ion peaks decreases, indicating reduced growth. Spectrum E5 shows impaired growth from a kanamycin concentration of 3.125 μg/mL. Spectrum D5 shows stronger impairment of growth from a kanamycin concentration of 12.5 μg/mL. Spectrum C5 shows no significant growth is observed from a kanamycin concentration of 50.0 μg/mL. Spectrum B5 likewise shows no significant growth is observed from a kanamycin concentration of 200.0 μg/mL.

In FIG. 2 can be seen a graph of the ratio of heavy/light LA ions from the spectra in FIG. 1, where light LA is measured as the intensity of the ion peak at 688.5 m/z and heavy LA is measured as the intensity of the ion peak at 692.5 m/z. In addition, FIG. 2 shows heavy/light LA calculations from a similar set of spectra using carbenicillin in place of kanamycin. As can be seen from FIG. 2, as will be appreciated by those skilled in the art, the relationship between heavy/light ratio and antibiotic concentration is not linear, but heavy/light ratio is lowest at the highest antibiotic concentrations, and heavy/light ratio is approximately 0:1 for the two highest kanamycin concentrations.

Thus it can be seen from FIGS. 1 and 2 that concentration of kanamycin and carbenicillin and inhibition by of P. fluorescens growth in 8% D₂O are correlated with the ratio of the intensity of undeuterated lipid ions to deuterated versions of the same ions, the aforementioned undeuterated and deuterated ions having been previously observed in P. fluorescens samples.

In at least one embodiment, a method of this embodiment 17 is used to determine growth inhibition of at least one cell, cell culture, tissue, microbial community, biofilm, or similar. In at least one embodiment a method of this embodiment 17 is used to determine growth inhibition of a sample from embodiments A-C. In at least one embodiment, a method of this embodiment 17 is used on a sample prepared according to a method of embodiments D, E or 1a-1e. In at least one embodiment, a method of this embodiment 17 is used to determine growth inhibition of a sample analyzed by a method of embodiments 2-4, 5a-5c, 6-10, 21, 22, 200-204, or 300. In at least one embodiment a method of this embodiment 17 is used to determine growth inhibition for the purpose of determining and/or measuring antibiotic resistance and/or susceptibility. In at least one embodiment a method of this embodiment 17 is used to determine growth inhibition for the purpose of determining and/or measuring one or more of an ADME-Tox parameter or other measure, efficacy, release profile, toxicity, toxicology, and/or drug treatment effect for at least one substance.

### Embodiment 18: Determining Growth, Growth Inhibition, and/or Response by Data-Independent Precursor Ion Selection Mass Spectrometry

In at least one embodiment, growth, growth inhibition, and/or response is determined and/or measured as in a method of embodiment 15, 17, and/or 20; however, measurement of at least one ion for determination of growth, growth inhibition, and/or response is performed via tandem MS or MSⁿ spectroscopy. Further, for the aforementioned at least one ion, for tandem MS or MSⁿ, at least one precursor mass or precursor mass range is determined in a data-independent fashion. For example, and without limiting the disclosure, similar to embodiment 15, in at least one embodiment, a sample known or suspected to contain E. coli is prepared by a method of embodiments 1a-1e then analyzed according to a method of embodiment 2, then the ratio of intensities of specific fragment ions in a tandem spectrum taken with a parent mass window from about 1797 to 1808 m/z is used to determine or estimate growth, growth inhibition, or response. Further, in at least one embodiment, a sample known or suspected to contain E. coli is prepared by a method of embodiments 1a-1e then analyzed according to a method of embodiment 2, the presence of specific fragment ions in a tandem spectrum with a parent mass of about 1808 m/z is used to determine or estimate growth, growth inhibition, or response. As one skilled in the art will appreciate, each species of Gram-negative bacteria has characteristic LA ions, and similar characteristic ions exist for Gram-positive bacteria and fungi, so that if the species of a sample is known, the characteristic deuterated and undeuterated ions will likewise in many cases be known. One skilled in the art will appreciate that, prior to or at least by the completion of many situations of growth determination and/or measurement and/or growth inhibition determination and/or measurement and/or response determination and/or measurement, at least one species in a sample is known. For example, and without limiting the disclosure, the species in a sample is usually determined by one or more methods prior to AST testing. In at least one embodiment, a spectrum obtained according to a method herein is used both to identify the species in a sample and to assess growth, growth inhibition, and/or response.

Furthermore, as can be seen from FIGS. 7 and 27, certain lipid ions are common to a wide range of microbes. In at least one embodiment, growth and/or response is determined and/or measured and at least one precursor mass or precursor mass range is derived from or includes at least one undeuterated and/or deuterated ion that is not characteristic of a single microbial or other species. In at least one embodiment, growth and/or response is determined and/or measured, at least one precursor mass or precursor mass range is derived from or includes at least one ion that is an undeuterated or deuterated ion of a molecule of one or more of the classes phosphatidylcholines (“PCs”), phosphatidylethanolamines (“PEs”), phosphoglycerols (“PGs”), lipopolysaccharide (“LPSs”), lipid As (“LAs”), sphingolipid (“SPs”), lipoteichoic acids (“LTAs”), and sterols (“STs”).

In at least one embodiment, growth, growth inhibition, and/or response of at least one sample containing at least one species and/or strain of organism is determined and/or measured via a method that includes data-independent precursor ion selection

### Embodiment 19: Determining Growth, Growth Inhibition, and/or Response by Data-Dependent Precursor Ion Selection Mass Spectrometry

In at least one embodiment, growth, growth inhibition, and/or response is determined and/or measured as in a method of embodiment 15, 17, 18, and/or 20; however measurement of at least one ion for determination of growth, growth inhibition, and/or response is performed via tandem MS or MSⁿ spectroscopy. Further, for the aforementioned at least one ion, for tandem MS or MSⁿ, at least one precursor mass or precursor mass range is determined in a data-independent fashion.

Considering methods of embodiment 18, one skilled in the art will appreciate that, for a given data-independent method of embodiment 18, at least one embodiment of this embodiment 19 comprises an equivalent data-dependent method, wherein a precursor mass or mass range is selected based on the presence of ions in a precursor spectrum.

In at least one embodiment, spectra are produced via MRM, an SRM method other than MRM such as without limitation CRM or PRM, or a data-dependent precursor ion selection technique other than SRM.

In at least one embodiment, deuteration is exploited by a data-dependent precursor ion selection strategy.

For example, and without limiting the disclosure, in at least one embodiment, at least one undeuterated ion peak is observed, then instead of or addition to data-dependent precursor ion selection on at least one mass or mass range including the at least one undeuterated ion peak, data-dependent precursor ion selection is performed on at least one mass or mass range including at least one expected deuterated ion peak. As a further example without limiting the invention in any way, in at least one embodiment, at least one deuterated ion peak is observed, then instead of or addition to data-dependent precursor ion selection on at least one mass or mass range including the at least one deuterated ion peak, data-dependent precursor ion selection is performed on at least one mass or mass range including at least one expected undeuterated ion peak. As one skilled in the art will appreciate, for many types of MS instruments, tandem spectra usually have a lower LOD than precursor spectra. For a particular chemical species and a particular mass spectrum, either deuterated or undeuterated ions of the chemical species will have higher intensity. In at least one embodiment, evidence of a deuterated or undeuterated precursor ion is used to collect tandem spectra where both deuterated and undeuterated fragment ions would be expected to be observed.

In at least one embodiment, growth, growth inhibition, and/or response of at least one sample containing at least one species and/or strain of organism is determined and/or measured via a method that includes data-dependent precursor ion selection

### Embodiment 21: Raman Spectroscopy

In at least one embodiment, Raman spectroscopy is used to measure growth, growth inhibition, and/or response, and/or to practice at least one method herein. In at least one embodiment, Raman spectroscopy comprises one or more of vibrational Raman spectroscopy, spontaneous Raman spectroscopy, enhanced Raman spectroscopy, Stokes Raman spectroscopy, Anti-Stokes Raman spectroscopy, enhanced Raman spectroscopy, surface-enhanced Raman spectroscopy, stimulated Raman spectroscopy, inverse Raman spectroscopy, coherent anti-Stokes Raman spectroscopy, resonance Raman spectroscopy, transmission Raman spectroscopy, Raman spectroscopy of micro-cavity substrates, or Raman spectroscopy combined with time-correlated photon counting.

For example, and without limiting the disclosure, in at least one embodiment, at least one sample prepared according to a method of embodiments D, E, or 1a-1e is analyzed via vibrational Raman spectroscopy. As a further example at least one sample is analyzed by illuminating the at least one sample with a 532 nm excitation laser, then the Raman shift spectrum is collected and C-D (carbon-deuterium) bonds appear as a peak in the range 2040-2300 cm⁻¹, and C—H (carbon-hydrogen) bonds are observed at as a peak in the range 2800-3100 cm⁻¹. The presence or absence in at least one Raman shift spectrum of a C-D peak, optionally conditioned on the presence of a C—H peak, is used in at least one embodiment to determine the presence or absence of growth and/or the inhibition of growth. Further, the ratio of the intensities of the aforementioned peaks is correlated with growth and in at least one embodiment is used to estimate growth, growth rate, and/or growth inhibition.

In at least one embodiment, Raman spectroscopy is performed using a simplified and consequently inexpensive Raman spectroscope consisting of at least a laser, a low-cost cut or band-pass filter, and a photodetector or inexpensive CCD is used to detect C-D bonds or C-D and C—H bonds.

In at least one embodiment, a Raman spectrometer is configured to measure a single Raman shift or a narrow range of Raman shift values by use of a simple bandpass filter instead of a mechanism such a monochromator or interferometer. In at least one further embodiment, a Raman spectrometer is configured to measure a Raman shift corresponding to C-D bonds.

In at least one embodiment, Raman spectroscopy is performed in conjunction with time-correlated single photon counting (TCSPC). In at least one embodiment, Raman spectroscopy performed in conjunction with TCSPC (“TCSPC Raman spectroscopy,” or “TCSPC-R”) uses an apparatus as described or similar to that described in Huang et al, “Time-Correlated Raman and Fluorescence Spectroscopy Based on a Silicon Photomultiplier and Time-Correlated Single Photon Counting Technique” (DOI:10.1366/12-06736). In at least one embodiment, TCSPC-R is performed using an apparatus as described or similar to an apparatus in the aforementioned paper by Huang et al, but the apparatus in the aforementioned at least one embodiment does not include a monochromator. For example, and without limiting the disclosure, in at least one embodiment, TCSPC-R is performed using an apparatus as described or similar to an apparatus in the aforementioned paper by Huang et al, except that the apparatus in the aforementioned at least one embodiment does not include a monochromator but does include at least one bandpass filter. In at least one embodiment, an apparatus for TCSPC-R in part comprises a pulsed laser, a bandpass filter, one or more photon detectors, and a signal acquisition and processing circuit; wherein in a further embodiment each of the aforementioned one or more photon detectors independently comprises a silicon photomultiplier, a photomultiplier tube, a hybrid photomultiplier, an avalanche photodiode, a charge-coupled device, or some other kind of photon detector.

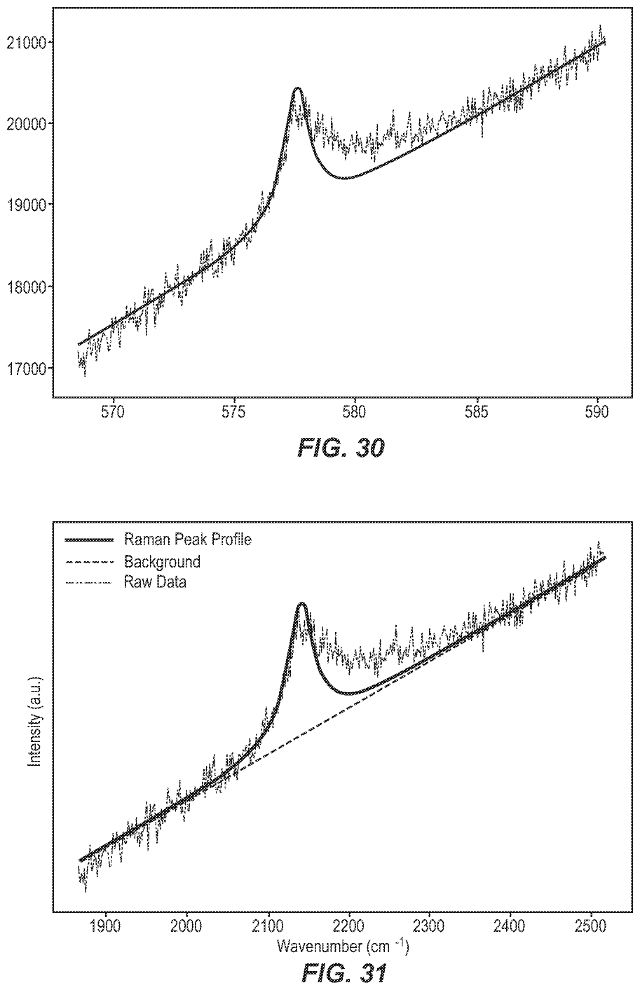

In FIGS. 30 and 31 is shown a Raman spectrum produced on a confocal Raman Microscope using an excitation laser with a wavelength of 514 nm. In FIG. 30, the Raman spectrum is expressed in wavelength. In FIG. 31 the spectrum is expressed in wavenumber. Shown in both figures is a Raman spectrum of a lipid extract from E. coli cultured in water containing about 8% deuterium (orange line). Shown in FIG. 31 is a linear estimate of background (dashed blue line). Shown in FIG. 30 is a Cauchy distribution with a location parameter of about 579 nm, added to linear estimated background. Shown in FIG. 31 is a Cauchy distribution with a location parameter of about 2135 cm⁻¹, added to linear estimated background. As will be appreciated by one skilled in the art, C-D bonds produce a Raman peak with a wavenumber of about 2135 cm⁻¹ corresponding to a Raman scattering peak wavelength of about 579 nm for an excitation wavelength of 514 nm.

### Embodiment 22: NMR Spectroscopy

In at least one embodiment, deuterium incorporation by organisms is measured by NMR, such as without limitation deuterium NMR, or hydrogen NMR. In at least one embodiment, carbon-deuterium bonds are measured by NMR, such as without limitation deuterium NMR, or hydrogen NMR.

### Embodiment 23: Spectroscopy Embodiments

In a preferred embodiment, H-D ratio indicates growth.

In a preferred embodiment, Single-cell Raman Spectroscopy, which is typically slow and requires various microfluidic apparatus, is not used.

In a preferred embodiment, a D₂O concentration is used that is less than 100%. In a further preferred embodiment, the D₂O concentration used is less than 30%. In a further preferred embodiment, the D₂O concentration used is less than 10%. The lower the D₂O concentration, the lower the cost for D₂O used as a reagent. Moreover, D₂O concentrations greater than 30% can cause alterations in the growth behavior of microorganisms, and some microorganisms do not grow well in D₂O concentrations greater than 30%.

In a preferred embodiment, at least one MIC value is determined. In a further preferred embodiment, determination of susceptible/resistant status and/or susceptible/intermediate/resistant status is based on at least one threshold value of at least one MIC value.

In at least one embodiment, determination of susceptible/resistant or susceptible/intermediate/resistant is based on at least one ratio of deuterated to undeuterated peaks in at least one spectrum and is not based on MIC values. In at least one embodiment, determination of susceptible/resistant or susceptible/intermediate/resistant is based on both on at least one ratio of deuterated to undeuterated peaks in at least one spectrum and at least one MIC value.

In a preferred embodiment, Raman spectroscopy is performed on a liquid sample without removing material from said liquid sample. In a preferred embodiment, Raman spectroscopy is performed on an incubating microbial sample while the microbial sample is incubating and/or in the volume in which the microbial sample is incubating or has incubated.

In a preferred embodiment, bacteria and/or other microorganisms are not immobilized on or in a surface. In a preferred embodiment, bacteria and/or other microorganisms are not bound to a binding partner. In a preferred embodiment, bacteria and/or other microorganisms are not bound in or on a detection surface. In a preferred embodiment, bacteria and/or other microorganisms are not reacted with an affinity reagent. In a preferred embodiment, bacteria and/or other microorganisms are not bound to a chemical label.

In at least one embodiment, a Raman spectrum is not compared to a reference Raman spectrum to identify microbial species or determine microbial growth. In at least one embodiment, features are extracted from a TCSPC-R spectrum that are correlated with microbial and/or cellular species and/or growth, and/or another analytical measure as described herein.

### Embodiment 100: Microorganisms Adapted to D₂O

It has been well known for some time that microorganisms can be progressively adapted to D₂O. (Richards 1934 PMCID: PMC533676) Bacteria typically adapt to pure D₂O via progressive adaptation to increasing D₂O concentrations in about ten passages, albeit with significant regulatory adaptations. Many species of bacteria can survive in 100% D₂O, even without progressive adaptation. Yeasts can be progressively adapted to typically about 30% D₂O composition, but typically can't adapt to pure D₂O.

In at least one embodiment, at least one microorganism is adapted to a specific concentration or range of concentrations of D₂O or to 100% concentration D₂O by progressively exposing the at least one microorganism to increasing concentrations of D₂O.

In at least one embodiment, at least one organism is adapted to a specific range of D₂O concentrations, by optionally adapting the at least one microorganism as described in this example to a first concentration or range of concentrations, then sequentially or sequentially and progressively exposing the at least one organism to at least two D₂O concentrations or to at least one D₂O concentration gradient.

In at least one embodiment, at least one polymicrobial specimen, microbial community, biofilm, and/or other combination of microbes is adapted by a method described herein to a specific concentration, to a range of concentrations, or to 100% concentration of D₂O.

### Embodiment 101: Generating Substances in D₂O

FIGS. 3-14 show that E. coli naturally produces PEs and PGs, and when E. coli is grown in water containing a concentration of D₂O, the straight chains of the aforementioned PEs and PGs will have an H-D ratio in equilibrium with the H-D ratio of the environment. FIGS. 16 and 21 show that when E. coli is grown in a deuterated environment, the straight chains of LAs produced will have an H-D ratio in equilibrium with the H-D ratio of the growth environment.

In at least one embodiment, one or more species and/or strains of a microorganism are optionally adapted to D₂O according to a method of embodiment 100 and the one or more species and/or strains of a microorganism are used to generate at least one of a lipid from embodiment F, another lipid, and/or another molecule, wherein the one or more strains and/or species of microorganisms are grown in at least one concentration of D₂O in H₂O at any value from 0% to 100% concentration of D₂O, and wherein at least one molecule produced by the one or more strains and/or species of microorganisms is deuterated. In at least one embodiment, a method of embodiments D or 1a-1c is used to extract a generated product from a microorganism.

In at least one further embodiment, at least one lipid in embodiment F, some other lipid, and/or some other molecule is generated, wherein the method of the previous example is used, but furthermore at least one metabolic altering or other bioengineering technique, comprising a gene editing, gene silencing, transfection, transcription modifying, or other molecular engineering technique is used to enable, suppress, up-regulate, and/or down-regulate at least one molecular pathway. In at least one further embodiment, the aforementioned at least one metabolic altering or other bioengineering technique is used in conjunction with at least one step-function change, pulse, gradient change, or other change in D₂O concentration, such that different metabolic pathways of a microorganism equilibrate to different H-D ratios. In at least one embodiment, at least one such different metabolic pathway equilibrates at certain hydrogen positions to an H-D ratio of 1:0, or 0% D. In at least one embodiment, at least one such different metabolic pathway equilibrates at certain hydrogen positions to an H-D ratio of 0:1, or 100% D. In at least one embodiment, at least one such different metabolic pathway equilibrates at certain hydrogen positions to an H-D ratio of a value greater than 0% D and less than 100% D. In this way, a product molecule is generated with different H-D ratios or probabilities at different locations of the molecule. For example, and without limiting the disclosure, in at least one embodiment, a product molecule with two fatty acid chains is generated with C—H bond positions 100% occupied by deuterium on one chain and by C—H bond positions less than 100% occupied by deuterium on the other chain.

In at least one further embodiment, at least one lipid from embodiment F, some other lipid, and/or some other molecule is generated, wherein the method of the previous example is used, except that at least one component of cellular media instead of or in addition to water is deuterated, said percentage possibly being any value including 100%.

In at least one embodiment, E. coli or a bacterium other than E. coli is used for a method herein. In at least one embodiment, a bacterial strain of the genus Acinetobacter, Bordetella, Burkholderia, Francisella, Pseudomonas, Salmonella, or Yersiniae is used for a method herein. In at least one embodiment a microbe from Aspergillus spp., Bacillus subtilis, Candida utilis, Clostridium acetobutylicum, Corynebacterium glutamicum, Penicillum, Saccharomyces spp., or Streptomyces spp. is used for a method herein.

In at least one embodiment, a microbial strain is used that has one or more of the following properties: expresses at least one non-endogenous biosynthesis enzyme, expresses one or more endogenous biosynthesis enzymes wherein the enzyme is modified, and/or has modified regulation of one or more endogenous biosynthesis enzymes. In at least one embodiment, at least one vector is delivered into a bacteria, the at least one vector optionally encoding at least one non-endogenous biosynthesis enzyme. In at least one embodiment, bacterial conjugation and/or another method of bioengineering is used to insert, modify, or modify the regulation of a biosynthesis enzyme. In at least one embodiment, at least one non-endogenous enzyme is used that is derived from a species or genus listed in this example or elsewhere herein. In at least one embodiment, at least one microbial strain expressing at least one enzyme is used, wherein the at least one enzyme is an endogenous or non-endogenous enzyme and the at least one enzyme is modified and/or has modified regulation or is unmodified.

In at least one embodiment at least one microbial strain expressing at least one endogenous or non-endogenous enzyme is used, wherein the at least one endogenous or non-endogenous enzyme is a LA biosynthesis enzyme, a LPS biosynthesis enzyme, and/or an enzyme of a precursor of LA and/or LPS.

### Embodiment 102: Deuterated Lipids

Chemicals containing D are described as generally having the same chemical properties as equivalent chemicals containing ¹H. However, the deuterium kinetic isotope effect causes reaction rates of D-containing molecules to be slower and in some cases different than the H-containing equivalent. This causes deuterated molecules to have different kinetics, and can cause deuterated drugs to have different pharmacokinetics. Some deuterated lipids are metabolized in the body more slowly than their undeuterated equivalents, and relatedly may be more resistant to oxidation and more stable in other ways (Kusher 1998 Can. J. Physiol. Pharmacol. 77: 79-88). Deuterated lipids can have different patterns of binding affinities for receptors compared to their undeuterated equivalents.

In at least one embodiment, at least one microorganism strain is used to produce at least one lipid that is partially or wholly deuterated. In at least one embodiment, the at least one lipid is an otherwise naturally occurring molecular structure produced by the at least one microorganism, except that Ds have been substituted for Hs. In at least one embodiment, the at least one lipid is not an otherwise naturally occurring structure produced by the at least one microorganism, and instead the at least one microorganism has been induced and/or engineered to produce a variant, mimetic, analog, or synthetic structure not normally produced by the at least one microorganism.

As can be seen from FIGS. 8-24, deuterated lipids are readily and precisely detected by MS and other means.

In at least one embodiment, at least one partially or wholly deuterated lipid described in this example is used for a non-therapeutic purpose or for a non-therapeutic aspect of a purpose, such as for example as a diagnostic or research tool.

In at least one embodiment, at least one organism is grown in at least one solution containing a concentration or concentration gradient of D₂O, producing one or more deuterated lipid chemical species. In at least one embodiment, the aforementioned one or more lipid chemical species exhibits at least one ADME-Tox or efficacy property of medical use that is distinct from the same property of the equivalent undeuterated chemical species.

In a preferred embodiment, microbial or other culture containing D₂O produces deuterated lipids at an attractive cost compared to other isotope-enriched manufacturing processes. D₂O can be purchased at present for about $6 a gram in high purity, or $1 a gram in purities sufficient for at least one embodiment of the present invention. In contrast, other heavy-labeled ingredients such as heavy-labeled culture media cost hundreds to thousands of dollars per gram. Furthermore, D₂O can be directly recycled from waste, but media components cannot at present be directly recycled. Lipids derived from some pathways in some microbial species, such as NADPH synthesis in E. coli, equilibrate to the H-D ratio of solution. Consequently, deuterating culture media components is unnecessary for at least some embodiments, and in at least one embodiment, deuterated solution achieves an effect not achievable through deuterated media. Bacteria produce many lipids of interest in large quantities, and methods described herein efficiently extract the lipids of interest from bacterial culture. Therefore, at least one embodiment of the present invention comprises a cost-efficient method of manufacturing deuterated lipids.

In at least one embodiment, a lipid mimetic is produced. The modifications in the mimetics may be of any kind, including modifications to the fatty acid content and/or number, the number of phosphates and/or modification thereof, and the number or type of sugar.

In at least one embodiment, a deuterated chemical described in this embodiment 102 is not strictly speaking a lipid or is not a lipid in the narrowest sense of the term. For example, and without limiting the disclosure, in at least one embodiment, an entire microorganism, or a microorganism cell wall skeleton is produced.

### Embodiment 103: Deuterated Lipid A

Lipid As (LAs), sometimes also referred to as “endotoxins” are used by pathogenic Gram-negative bacteria to manipulate the host immune system and environment. LAs bind with varying affinity to receptors including the TLR4 complex and TLR2. By agonizing or antagonizing these receptors, LAs provoke or suppress inflammatory responses. Consequently, LAs and LA analogues have been used or proposed for medicinal purposes including as adjuvants to provoke an immune response in vaccines as well as anti-inflammatories to combat sepsis. A TLR4 antagonist can reduce inflammation and act as an anti-inflammatory agent and/or sepsis treatment. A TLR4 agonist can provoke inflammation and act as an adjuvant. Furthermore, TLR2 agonists also act as adjuvants.

Deuterated LAs offer potential advantages over their undeuterated equivalents. Due to kinetic isotope effects, deuterated LAs are typically more resistant to oxidation, have lower metabolism and/or have longer biological half-life as compared to their undeuterated equivalents. The deuterium kinetic isotope effect, the shorter C-D bond length compared to the C—H bond length, and other chemical differences cause deuterated lipids to have different and potentially beneficial binding kinetics compared to their undeuterated equivalents.

Deuterated adjuvants offer the additional potential advantage of greater shelf life. The C-D bond is as much as ten times stronger than the C-′H bond, and many deuterated lipids are more stable than their undeuterated equivalents. This increased stability is valuable in many applications, including in medicines generally, but especially in vaccines, where longer room-temperature stability is often desired.

In at least one embodiment, at least one partially or wholly deuterated LA agonizes or antagonizes at least one of TLR2, TLR4, or another toll-like receptor, or another immune-related receptor. Furthermore, in at least one embodiment, the at least one partially or wholly deuterated LA is generated according to a method of embodiment 101.

In at least one embodiment at least one partially or wholly deuterated LA is used for a purpose not described in embodiment 102.

In at least one embodiment, at least one partially or wholly deuterated LA is used for a purpose described in embodiment 102 or embodiment 103.

As can be seen for example in FIGS. 15, 16, 20, and 21, in at least one embodiment of this invention deuterated lipid A molecules are observed.

In at least one embodiment, a deuterated chemical described in this embodiment 103 is not strictly speaking an LA or is not an LA in the narrowest sense of the term. For example, and without limiting the disclosure, in at least one embodiment an LA mimetic is produced and used for a purpose described herein. As a further example without limiting the invention in any way, in at least one embodiment an LPS (lipopolysaccharide) and/or an LOS (lipooligosaccharide) optionally containing or bound to an LA or an LA mimetic is produced and used for a purpose described herein.

### Embodiment 104: Deuterated LTA

Gram-positive bacteria produce LTAs, which can play roles partially analogous to LAs in Gram-negative bacteria. The therapeutic, diagnostic, and research potential of LTAs has been given little consideration, and relatedly, the biology of the Gram-positive membrane and of LTA is poorly understood when compared to that of Gram-negative bacteria and LAs.

In at least one embodiment, at least one undeuterated, partially deuterated, or wholly deuterated LTA agonizes or antagonizes at least one of the receptors TLR2, TLR4, or another toll-like receptor, or another immune-related receptor. Furthermore, in at least one embodiment, the at least one undeuterated, partially deuterated, or wholly deuterated LTA is generated according to a method of embodiment 101. In at least one embodiment at least one partially or wholly deuterated LTA is used for a purpose, in a manner, or in an embodiment equivalent to that described in embodiments 103 or 102.

In at least one embodiment, a deuterated chemical described in this embodiment 104 is not strictly speaking an LTA or is not an LTA in the narrowest sense of the term.

### Embodiment 105: Modified Materials

In at least one embodiment, at least one substance produced by a method of embodiments 100-104 or by another method herein is formulated and/or chemically modified by means familiar to those skilled in the art, then the aforementioned chemically modified at least one substance is used for at least one purposed described in embodiments 100-104 or at least one purpose described elsewhere herein.

For example, and without limiting the disclosure, in at least one embodiment, LAs produced by the method of embodiment 103 are formulated with other materials into an emulsion, and the emulsion is used as a vaccine adjuvant. As a further example without limiting the invention in any way, in at least one embodiment, at least one substance produced by a method herein is formulated into a composition in a salt, neutral, or free base form.

### Embodiment 200: Antimicrobial Susceptibility

In at least one embodiment, at least one sample as described in embodiments A-C or elsewhere herein is tested for antimicrobial susceptibility and/or antimicrobial resistance by means of (a) incubating or culturing the at least one sample in at least one environment, wherein each at least one environment contains at least a concentration of D₂O and a concentration of an antimicrobial agent, producing at least one inoculum; (b) preparing the at least one inoculum for analysis, including without limiting the invention in any way using any method of embodiments D, E, or 1a-1c; (c) analyzing the prepared inoculum, including without limiting the invention in any way analyzing using any method of embodiments 2-4, 5a-5c, 6-10, and 21.

In at least one embodiment, analysis is by a Bruker autoflex MALDI mass spectrometer and the aforementioned concentration of D₂O is between about 3% and 8% by molarity. Below a concentration of 50% deuterium, as the concentration of D₂O decreases, the variance in the number of incorporated deuterium atoms decreases, resulting in progressively greater relative abundances for a progressively smaller number of deuterated ions, and consequently higher signal-to-noise-ratio for deuterated ion peaks and easier detection. However, the mean mass shift of deuterated molecules also decreases as D₂O concentration decreases, eventually making the aforementioned deuterated ion peaks indistinguishable from undeuterated ion peaks, thereby limiting the lowest desirable D₂O concentration or equivalently H-D ratio for a given analytical task. In the case of detecting growth for antimicrobial susceptibility testing (AST) using the aforementioned MALDI mass spectrometer operated with a specific set of control parameters, the aforementioned lowest desirable D₂O concentration is between 3% and 8% by molarity.

### Embodiment 201 Antimicrobial Susceptibility Test

In at least one embodiment, at least one sample according to embodiments A-C or elsewhere herein is analyzed by analogy to an AST assay using an MIC procedure of a type familiar to those skilled in the art, by means of (a) incubating or culturing the at the least one sample in multiple environments, each such environment containing at least a concentration of D₂O and a concentration of an antimicrobial agent selected from a set of such concentrations, each aforementioned environment thereby producing an inoculum; (b) preparing each inoculum for analysis, including without limiting the invention in any way using any method of embodiments D, E, or 1a-1c or any appropriate method herein; (c) analyzing the prepared inoculum, including without limiting the invention in any way analyzing using any method of embodiments 2-4, 5a-5c, 6-10, or 21, or any appropriate method herein.

In a preferred embodiment, at least one environment has an antimicrobial concentration of 0% of the antimicrobial agent. In at least one embodiment, the aforementioned at least one environment with an antimicrobial concentration of 0% serves as a control. In at least one embodiment, more than one such environment with an antimicrobial concentration of 0% is used, to easily allow for multiple sterile checks for growth of the sample over time, so that analysis of the environments which contain nonzero concentrations of an antimicrobial agent are performed only after growth of the sample has been detected in at least one of the aforementioned multiple volumes. In at least one embodiment, lipids from at least one environment serving as a control are evaluated at two or more different times. In at least one embodiment, lipids from at least one environment not serving as a control are evaluated at two or more different times.

In at least one embodiment, the D₂O concentrations in different environments are not all the same. In at least one embodiment, at least one environment has a 0% D₂O concentration and 0% concentration of any antimicrobial agent, said at least one environment serving as a control for undeuterated ion peaks. The aforementioned control allowing more accurate measurement of growth in a deuterated environment.

In at least one embodiment, multiple antimicrobial agents are distributed to multiple environments as described above in this example, similar to conventional microdilution AST tests. For example, and without limiting the disclosure, in at least one embodiment, a 96-well microtiter plate contains 17 groups of 5 wells per group to analyze 17 antimicrobial agents. In each group, each antimicrobial agent is present at 5 different concentrations. This leaves 11 volumes as deuterated controls containing no antimicrobial agent. Once per hour, a sample is prepared from one control well using a method of embodiments D, E or 1a-1c or any appropriate method herein; and analyzed by any method of embodiments 2-4, 5a-5c, 6-10, or 21, or any appropriate method herein. In at least one embodiment at least one antimicrobial agent is present at a different number of concentrations than at least one other antimicrobial agent. For example, and without limiting the disclosure, in at least one embodiment, multiple antimicrobial agents are distributed to multiple culture volumes as described above in this example, and at least one antimicrobial agent is distributed to a different number of culture volumes than at least one other antimicrobial agent.

When growth above a threshold value is detected by deuterated peaks in one of the aforementioned 11 volumes, samples from the volumes containing multiple antimicrobial agents at multiple concentrations are prepared using a method of embodiments D, E or 1a-1c or any appropriate method herein and analyzed by any method of embodiments 2-4, 5a-5c, 6-10, or 21, or any appropriate method herein. From spectra and/or other data produced from analysis of the volumes containing multiple antimicrobial agents at multiple concentrations, MICs and/or susceptibility status is measured, estimated, and/or determined for at least one species and/or strain in the aforementioned at least one sample, for each of the multiple antimicrobial agents.

Additionally and optionally, at any time or at multiple times during the process of this embodiment, from the aforementioned spectra and/or other data, and/or analysis of the aforementioned controls, additional information about the sample, such as without limiting the invention in any way the species, genus, taxonomic level above species, strain, clone, phenotype and/or Gram stain status of at least one organism in the sample is also determined from analysis of one or more spectra. For example, and without limiting the disclosure, in at least one embodiment, additional information is determined by MALDI protein fingerprinting and/or by BACLIB lipid fingerprinting. As a further example without limiting the invention in any way, in at least one Gram-negative bacteria species, taxon other than species, strain, clone, or subpopulation present in the at least one sample, modification of at least one LA chemical species for example by the addition of phosphoethanolamine and/or aminoarabinose is detected, where said modification is associated with resistance or susceptibility to at least one antimicrobial, antimicrobial class, or antimicrobial combination.

### Embodiment 202: Apparatus for AST or Similar

In at least one embodiment is used an apparatus containing a multiplicity of wells or volumes (“wells”), suitable for a method of embodiment 201 or any appropriate method herein. In at least one embodiment, one or more of the multiplicity of wells contains dried and/or lyophilized material including at least one antimicrobial agent at a range of concentrations. Alternatively or in addition, in at least one embodiment, an antimicrobial agent is not pre-supplied in one or more of the multiplicity of wells, and the user of the apparatus adds their own antimicrobial or similar or other inhibitor agent, toxin, or other material or no material at one or more concentrations in at least one well, allowing the user of the apparatus to evaluate growth for one or more materials and/or conditions of the user's choosing for at least one organism in at least one sample.

In at least one embodiment, microbiological media such as without limitation bacterial growth media, Mueller-Hinton broth, blood agar, potato dextrose broth, some other broth or agar, or some other growth media is part of an AST apparatus. In at least one embodiment, to said microbiological media is added water containing an amount of deuterium, measured as a percentage of D₂O by molarity, or relatedly a similar ratio of H to D in solution.

In at least one embodiment, an AST apparatus includes a container holding a mixture of H₂O and D₂O, and another container holding dehydrated and/or lyophilized media, and/or dehydrated and/or lyophilized media is contained in one or more of the multiplicity of wells or any multiplicity of wells of the apparatus. In at least one embodiment, the mixture of H₂O and D₂O is added to a container holding dehydrated and/or lyophilized media, and/or to at least one well. In at least one embodiment a solution of media in a mixture of H₂O and D₂O is added to a container containing optionally dehydrated, dried, and/or lyophilized antibiotics. In at least one further embodiment, the aforementioned solution has been inoculated with a microbial species, whereas in at least one other further embodiment the aforementioned solution has not been inoculated with a microbial species.

In at least one embodiment a ramification, manifold, or similar means of distributing a sample to a multiplicity of wells is provided.

### Embodiment 203: Apparatus for AST or Similar

In at least one embodiment, a method of embodiment 202 or another appropriate method herein is performed using an apparatus consisting of at least a microtiter plate, at least one flat plate such as a MALDI plate, and a pipettor or similar liquid-handling device.

1. Wells of the microtiter plate are prepared to contain a microbial sample, growth media, and a quantity of deuterated water. One or more antimicrobial agents, toxins, growth inhibitors, or other substances is introduced into one or more wells of the microtiter plate at one or more concentrations.

2. Optionally, at least one time at equal or unequal time intervals, a portion or the entire contents of at least one well not containing any aforementioned antimicrobial agent, toxin, growth inhibitor, or other substance is transferred such as via pipette to a MALDI plate, and/or the portion or entire contents of at least one well is analyzed by a method of embodiments D, E, or 1a-1c followed by a method of embodiments 2-4, 5a-5c, 6-10 or 21, such analysis performed to determine the degree to which the sample in the well has grown. If the sample has sufficiently grown, proceed to step 3.

3. A portion or the entire contents of at least one well is transferred such as via pipette to a MALDI plate, and/or the portion or entire contents of at least one well is analyzed by a method of embodiments D, E, or 1a-1c followed by a method of embodiments 2-4, 5a-5c, 6-10, or 21.

4. For each well analyzed, using ratios of undeuterated to deuterated peaks, or any mathematical model or function of growth under the conditions of this example, the amount of growth in each well of interest is estimated.

5. Optionally, for wells containing the same antimicrobial agent or other substance at different concentrations, an MIC is estimated by interpolating and/or projecting the curve formed by the growth at different concentrations, or by any mathematical model of MIC from growth rates as obtained herein.

### Embodiment 204: Apparatus for AST or Similar

In at least one embodiment, a method of embodiment 202 or another appropriate method herein is performed using an apparatus consisting of at least a microtiter plate.

1. Wells of the microtiter plate are prepared as in step 1 of embodiment 203.

2. Optionally, at least one time at equal or unequal time intervals, a portion or the entire contents of at least one well not containing any aforementioned antimicrobial agent, toxin, growth inhibitor, or other substance is transferred such as via pipette and prepared according to a method of embodiments D, E, or 1a-1c, or alternatively prepared in-place according to a method of embodiments D, E or 1a-1c; and the portion or entire contents of at least one well is the analyzed by a method of embodiments 2-4, 5a-5c, 6-10, or 21, such analysis performed to determine if the sample in the well has grown sufficient to proceed.

3. A portion or the entire contents of at least one well is transferred such as via pipette and prepared according to a method of embodiments D, E or 1a-1c, or alternatively the portion or entire contents of at least one well is prepared in-place according to a method of embodiments D, E, or 1a-1c; and the portion or entire contents of at least one well is then analyzed by a method of embodiments 2-4, 5a-5c, 6-10, or 21.

4. For each well analyzed, using ratios of undeuterated to deuterated peaks, or any mathematical model or function of growth under the conditions of this example, the amount of growth in each well of interest is estimated.

5. Optionally, for wells containing the same antimicrobial agent or other substance at different concentrations, an MIC is estimated by interpolating and/or projecting the curve formed by the growth at different concentrations, or by any mathematical model of MIC from growth rates as obtained herein.

### Embodiment 300: AST with Combined Raman Spectroscopy and Mass Spectrometry

In at least one embodiment, at least one method of embodiments 3, 4, or 21 is performed in conjunction with at least one method of embodiments 200-201 to test at least one sample as described herein for antimicrobial susceptibility and/or antimicrobial resistance by combining the methods of Raman spectroscopy and mass spectrometry.

For example, and without limiting the disclosure, in at least one embodiment, a method using an apparatus with multiple volumes and/or wells as described in embodiment 200 is used for AST. Further, growth of at least one well is measured via Raman spectroscopy to determine that sufficient growth has occurred for AST determination and/or measurement, then growth and/and or growth inhibition of one, some, or all wells of the aforementioned apparatus is determined via mass spectrometry, using any appropriate method herein.

In at least one embodiment, a method of this embodiment 300 is performed using at least one apparatus described in embodiments 202-204.

In at least one embodiment, Raman spectroscopy is performed as in this example using a simplified and consequently inexpensive Raman spectroscope consisting of a low-cost cut filter and a photodetector or inexpensive CCD is used to detect C-D bonds or C-D and C—H bonds.

### Embodiment 400: TCSPC Analysis

In at least one embodiment, an apparatus as described herein is used for an analysis other than AST or microbial ID. In a preferred embodiment, a TCSPC platform is used for a variety of analytical tasks.

### Embodiments 501-527

501. A method for detecting and/or measuring growth of at least one microbial population in a sample containing or suspected to contain at least one microbe, comprising: (a) incubating all or a portion of the sample in an environment wherein the concentration of deuterium in liquid solution has been increased above natural abundance; (b) one or more times, applying all, a portion of, or a substance extracted from the environment containing the sample to a mass spectrometer, producing one or more mass spectra; (c) in the one or more mass spectra, determining the presence of one or more peaks representing deuterated and/or undeuterated ions; (d) detecting growth as at least one of (i) the presence of at least one deuterated ion, (ii) the presence of at least one deuterated ion and one corresponding undeuterated ion, (iii) the presence of at least one pattern of deuterated ions, or (iv) the presence of at least one pattern of deuterated ions and at least one pattern of corresponding undeuterated ions; and (e) optionally measuring the relative or absolute degree of growth of the at least one microbial species in the sample as the ratio of at least one deuterated ion to at least one undeuterated ion and/or the ratio of at least one deuterated ion pattern to at least one undeuterated ion pattern.

502. A method for determining the growth inhibition or acceleration effect on a sample of at least one chemical agent by applying the method of embodiment 501 to one or more subsamples of the sample, wherein the at least one chemical agent is added in one or more concentrations to some or all of the one or more subsamples, and growth inhibition or acceleration is determined as at least one of (a) the minimum inhibitory concentration of the at least one chemical agent at which growth is not observed; (b) the minimum inhibitory concentration of the at least one chemical agent calculated from growth measurements at one or more concentrations and one or more points in time; or (c) growth rate calculated from growth measurements at one or more concentrations and one or more points in time.

503. The method according to any one of embodiments 501 or 502, wherein instead of or in addition to the at least one microbial population, growth of at least one cellular type that is not a microbe is evaluated.

504. The method according to embodiment 502, wherein the at least one chemical agent is an antimicrobial agent, antimicrobial class, or antimicrobial combination, and the determination of growth is used to determine antimicrobial susceptibility or resistance and/or an antibiogram of at least one of (a) the at least one microbial population; (b) at least one microbial species, taxonomic classification above the level of species, taxonomic classification below the level of species, strain, Gram stain classification, clone, or phenotype present or suspected to be present in the at least one microbial population; or (c) a communal or cooperative resistance of two or more microbial species, taxonomic classification above the level of species, taxonomic classification below the level of species, strain, Gram stain classification, clone, or phenotype present or suspected to be present in the at least one microbial population.

505. The method according to any one of embodiments 501-504, wherein (a) at least one mass spectrometer is used that is an electrospray mass spectrometer, a desorption electrospray ionization mass spectrometer, a time-of-flight mass spectrometer, a quadrupole mass spectrometer, a triple quadrupole mass spectrometer, a magnetic sector mass spectrometer, an ion trap mass spectrometer, a quadrupole trap mass spectrometer, an orbitrap mass spectrometer, a gas chromatograph mass spectrometer, a matrix-assisted laser desorption/ionization (“MALDI”) mass spectrometer, a Time-of-Flight Secondary Ion Mass Spectrometry (“TOF-SIMS”) mass spectrometer, an ion mobility mass spectrometer, a plasma chromatograph, an inductively-coupled plasma mass spectrometer, a mass cytometer, an accelerator mass spectrometer, a Fourier transform mass spectrometer, a Fourier-transform ion cyclotron resonance mass spectrometer, a mass spectrometer using an ambient ionization method such as direct analysis in real time, a mass spectrometer using surface acoustic wave nebulization or another nebulization technique, a mass spectrometer using Rapid Evaporative Ionization Mass Spectrometry, or another type of mass spectrometer; and (b) at least one of the one or more mass spectra was produced via a parent ion scan, tandem ion scan, MSⁿ scan, data-independent precursor ion selection, data-dependent precursor ion selection, multiple reaction monitoring (“MRM”), selected reaction monitoring (“SRM”), consecutive reaction monitoring (“CRM”) parallel reaction monitoring (“PRM”), or another mass spectrometry technique.

506. The method according to any one of embodiments 501-505, wherein (a) the amount of deuterium in at least one environment is greater than the natural abundance of deuterium by more than 20%, about 20%, between 20% and 10%, about 10%, between 10% and 8%, about 8%, between 8% and 5%, about 5%, between 5% and 3%, about 3%, or less than 3%; and (b) optionally, the amount of deuterium in at least one environment is equal to the natural abundance of deuterium, corresponding to an increase above natural abundance of 0%.

507. The method according to any one of embodiments 501-506, wherein incubation comprises one or more time intervals, wherein at each of the one or more time intervals, incubation is at a temperature for a period of time, and (a) for at least one of the one or of the time intervals, incubation temperature is less than 25° C., about 25° C., between 25° C. and 30° C., about 30° C., between 30° C. and 35° C., about 35° C., between 35° C. and 37° C., about 37° C., or greater than 37° C. and (b) for at least one of the time intervals, incubation time is less than 30 minutes, about 30 minutes, between 30 minutes and 1 hour, about 1 hour, between 1 and 2 hours, about 2 hours, between 2 and 3 hours, about 3 hours, between 3 and 4 hours, about 4 hours, between 4 and 5 hours, about 5 hours, between 5 and 6 hours, about 6 hours, between 6 and 7 hours, about 7 hours, between 7 and 10 hours, about 10 hours, between 10 and 16 hours, about 16 hours, between 16 and 24 hours, about 24 hours, between 24 and 48 hours, about 48 hours, or greater than 48 hours.

508. The method according to any one of embodiment 501-507, wherein at least one deuterated or undeuterated ion is an ion is a product of at least one of a membrane lipid, a phospholipid, a lipid A, a lipopolysaccharide, a phosphatidylcholine, a phosphatidylethanolamine, a phosphoglycerol, a cardiolipin, a lipoteichoic acid, a sphingolipid, a sterol, or a member of phospholipid species PE29:1, PE29:2, PE29:3, PE30:1, PE30:2, PE30:3, PE31:1, PE31:2, PE31:3, PE32:1, PE32:2, PE32:3, PE33:1, PE33:2, PE33:3, PE34:1, PE34:2, PE34:3, PE35:1, PE35:2, PE35:3, PG30:1, PG30:2, PG30:3, PG31:1, PG31:2, PG31:3, PG32:1, PG32:2, PG32:3, PG33:1, PG33:2, PG33:3, PG34:1, PG34:2, PG34:3, PG35:1, PG35:2, PG35:3, PG36:1, PG36:2, or PG36:3.

509. The method according to any one of embodiment 501-508, wherein lipids are extracted for analysis from at least one organism or at least one environment containing at least one sample by the fast lipid assay test method, the “BACLIB” method considered as a lipid extraction method, the “Caroff” method, the Bligh-Dyer method, the Folch method, the hot phenol method, two-phase extraction, or the TAO method (Liang 2018 doi.org/10.1021/acs.analchem.8b02611).

510. The method according to any one of embodiments 501-508, wherein at least one substance is extracted from all or a portion of the environment containing the sample by a method comprising:
placing a quantity of liquid from the environment in contact with at least one surface, wherein the at least one surface is optionally made of stainless steel or some other steel;
optionally enclosing the at least one surface to facilitate control of evaporation of the quantity of liquid, and furthermore optionally placing a quantity of liquid with the at least one surface to further facilitate control of the evaporation of the quantity of liquid;
optionally adding a solution of citric acid and sodium citrate or another wet or dry buffer or acidic composition to the at least one surface and/or to the quantity of liquid;
optionally heating the quantity of liquid on the at least one surface;
optionally washing the at least one surface;
optionally allowing the at least one surface to at least partially dry; and
optionally applying a composition comprising at least one solvent and an optional substance having the property of acting as a MALDI matrix.

511. The method according to embodiment 510, wherein the quantity of liquid is heated for less than 5 minutes, between 5 minutes and 15 minutes, about 15 minutes, about 20 minutes, about 25 minutes, about 30 minutes, or longer than 30 minutes.

512. The method according to embodiment 511, wherein the heating comprises heating the quantity of liquid in contact with the at least one surface to less than 80° C., about 80° C., between 80° C. and 95° C., about 95° C., about 100° C., about 110° C., about 121° C., about 125° C., about 130° C., about 135° C., or more than 135° C.

513. The method according to any one of embodiments 501-508, wherein lipids are extracted for analysis from at least one organism or at least one environment containing at least one sample by a method comprising:
placing a quantity of liquid from the environment in a separation column containing at least one solvent and/or a solvent gradient;
extracting lipids from at least one organism into solution in the column;
eluting lipids from the column into a mass spectrometer.

514. The method according to any one of embodiments 501-513, wherein the mass spectrometer optionally is in a negative ionization mode, and at least one spectrum is collected with a lowest m/z of less than 100, about 100, about 300, about 400, about 600, about 700, about 800, about 1000, or greater than 1000 m/z and a highest m/z of less than 200, about 200, about 300, about 400, about 600, about 700, about 800, about 900, about 1000, about 1200, about 1500, about 1800, about 2000, about 2200, about 2400, about 2500 or greater than 2500 m/z.

515. The method according to any one of embodiments 501-514, wherein in place of or in addition to applying all, a portion of, or a substance extracted from the environment containing the sample to a mass spectrometer, all, a portion of, or a substance extracted from the environment containing the sample is applied to another analytical instrument operating by means of laser induced fluorescence spectroscopy, atomic absorption spectroscopy, atomic emission spectroscopy, flame emission spectroscopy, acoustic resonance spectroscopy, cavity ring down spectroscopy, circular dichroism spectroscopy, Raman spectroscopy, surface enhanced Raman spectroscopy, coherent Raman spectroscopy, vibrational Raman spectroscopy, spontaneous Raman spectroscopy, enhanced Raman spectroscopy, Stokes Raman spectroscopy, Anti-Stokes Raman spectroscopy, enhanced Raman spectroscopy, surface-enhanced Raman spectroscopy, stimulated Raman spectroscopy, inverse Raman spectroscopy, coherent anti-Stokes Raman spectroscopy, resonance Raman spectroscopy, transmission Raman spectroscopy, Raman spectroscopy of micro-cavity substrates, Raman spectroscopy combined with time-correlated photon counting, cold vapor atomic fluorescence spectroscopy, nuclear magnetic resonance spectroscopy, electrical impedance spectroscopy, electron phenomenological spectroscopy, electron paramagnetic resonance spectroscopy, Fourier-transform spectroscopy, laser-induced breakdown spectroscopy, photoacoustic spectroscopy, photoemission spectroscopy, photothermal spectroscopy, spectrophotometry, vibrational circular dichroism spectroscopy, gamma spectroscopy, NMR spectroscopy, flow cytometry, or some other type of spectroscopy; or by means of a scintillation detector, scintillation counter, Geiger counter, ionization chamber, gaseous ionization detector, or other radiation detector.

516. The method according to any one of embodiments 501-515, wherein additionally the presence, quantity, taxon, or other property of at least one microbial organism in the sample is determined using at least one spectrum produced by the mass spectrometer and/or other analytical instrument;
wherein the taxon or the other property of the at least one microbial organism is a bacterial species; a yeast and/or fungal species; a microbial species other than bacteria, a yeast, or a fungi; a microbial strain; a microbial phenotype of resistance, intermediate resistance, or susceptibility to one or more antimicrobial agent, antimicrobial class, or antimicrobial combination; a microbial taxonomic classification above the level of species; a microbial taxonomic classification below the level of species, a Gram stain classification, or a clone;
wherein optionally, the presence, quantity, taxon, or other property of at least one microbial organism in the sample is determined by the BACLIB method.

517. The method according to any one of embodiments 501-516, wherein the sample comprises or is derived from a culture plate colony or smear, a broth culture sample, a blood culture sample, a sample from a biofluid, a clinical sample, a nonclinical sample, an environmental sample, a veterinary sample, an agricultural sample, a food or food safety sample, an industrial sample, a process control sample, a forensic sample, or a biofluid from a human or non-human source.

518. The method according to any one of embodiments 501-517, wherein the sample comprises or is derived from a urine specimen, a blood sample, a sample incubated in a blood bottle, sputum, a sample obtained from sputum, urine, feces, wound effluent, mucus, buccal swab, nasal swab, vaginal swab, nipple aspirate, sweat, saliva, semen or ejaculate, synovial fluid, bronchoalveolar lavage, endotracheal aspirate, tears, a urinary catheter sample, a culture plate, or another clinical or medical sample, or another human or mammalian material.

519. The method according to any one of embodiments 501-518, wherein the material applied to a mass spectrometer and/or other instrument has a volume of less than about 0.2 μL, about 0.2 μL, about 0.3 μL, about 0.4 μL, about 0.5 μL, about 1.0 μL, about 1.5 μL, about 2 μL, about 3 μL, about 4 μL, about 6 μL, about 8 μL, about 10 μL, about 20 μL, or greater than 10 μL.

520. The method according to any one of embodiments 501-519, wherein at least one measurement of growth is used to diagnose, predict, or predict the progress of a human or non-human disease; or to select or estimate the efficacy of a treatment for a human or non-human disease.

521. A kit for use in practicing a method according to one or more of embodiments 1-20, the kit comprising any one or more of:
an analytical instrument, optionally a spectrometer;
a plate, wherein the plate optionally comprises a material disposed on at least a portion of at least one surface of the plate;
a gasket, wherein the gasket comprises at least one hole having a shape and a size configured for placement over and alignment with at least one structured spot on the plate;
a solvent, wherein the solvent comprises any one or more of a buffer solution, an acidic solution, and a solid to which a liquid can be added to form a buffer or an acidic solution;
an enclosure, wherein the enclosure comprises a holder base, optionally the holder base comprises a reservoir, and a lid; and
a computing device or system comprising a software configured to receive and/or display spectroscopic data.

522. A deuterated PC, PE, PG, LPS, LA, SP, LTA, or ST or other lipid or a deuterated mimetic of a PC, PE, PG, LPS, LA, SP, LTA, or ST or other lipid.

523: A method of generating a deuterated lipid or deuterated lipid mimetic, comprising the steps of:
obtaining a bacterial strain that optionally has one or more of the following properties: expresses one or more non-endogenous lipid biosynthesis enzymes; expresses one or more endogenous lipid biosynthesis enzymes, wherein the enzyme is modified; and/or has modified regulation of one or more endogenous lipid biosynthesis enzymes;
optionally passaging the bacterial strain one or more times in one or more environments with deuterated solution;
subjecting the strain to conditions suitable for production of a lipid composition, in an environment with deuterated solution.

524: The method according to any one of embodiments 522 or 523, wherein the deuterated lipid or deuterated lipid mimetic is a lipooligosaccharide/lipid A-based mimetic.

525: A method of enhancing an immune response in a subject, comprising administering to the subject an effective amount of a deuterated lipid or deuterated lipid mimetic generated according to the method of embodiment 523 or by another method.

526. An immunomodulating lipid that is a deuterated lipid or deuterated lipid mimetic according to embodiment 522.

527. A method of treating or preventing sepsis in a subject, comprising administering to the subject an effective amount of an immunomodulating lipid according to embodiment 526.

### Embodiments 601-627

601. A method for detecting growth of at least one microbial population and/or growth of at least one non-microbial cellular population in a sample, comprising:

(a) incubating a sample containing or suspected to contain the at least one microbial population in an environment, wherein the environment comprises deuterium in a liquid solution at a concentration that is above a natural abundance of the deuterium in the liquid solution;

(b) extracting all or a portion of a substance from the environment and applying the all or a portion of the substance to a mass spectrometer;

(c) producing one or more mass spectra using the mass spectrometer; and

(d) detecting growth of the at least one microbial population in the sample by identifying one or more peaks representing any one or more of: (i) the presence of at least one deuterated ion; (ii) the presence of at least one deuterated ion and one undeuterated ion; (iii) the presence of at least one pattern of deuterated ions; (iv) the presence of at least one pattern of deuterated ions and at least one pattern of undeuterated ions; or (v) the presence of at least one pattern of deuterated and undeuterated ions.

602. The method of embodiment 601, further comprising measuring a relative or absolute degree of growth of the at least one microbial population in the sample as (1) the ratio of at least one deuterated ion to at least one undeuterated ion and/or the (2) ratio of at least one deuterated ion pattern to at least one undeuterated ion pattern.

603. The method of embodiment 601 or 602, wherein the sample comprises one or more subsamples and wherein the method further comprises, prior to or during the incubating in (a):
exposing one or more of the subsamples to at least one chemical agent at one or more concentration; and
identifying a growth inhibitory effect or a growth enabling and/or acceleration effect of the at least one chemical agent as: (1) a minimum concentration of the at least one chemical agent at which growth is not observed; (2) a minimum concentration of the at least one chemical agent calculated from growth measurements at one or more concentrations and one or more points in time; or (3) a growth rate calculated from growth measurements at one or more concentrations and one or more points in time

604. The method according to any one of embodiments 601-603, wherein growth of at least one non-microbial cellular population is detected.

605. The method according to embodiment 603 or 604, wherein the at least one chemical agent is an antimicrobial agent, antimicrobial class, or antimicrobial combination.

606. The method according to embodiment 605, further comprising, using the detected growth to (1) determine antimicrobial susceptibility or resistance of, and/or (2) generate an antibiogram of, at least one of (a)-(c):
(a) the at least one microbial population; (b) at least one microbial species, taxonomic classification above the level of species, taxonomic classification below the level of species, strain, Gram stain classification, clone, or phenotype present or suspected to be present in the at least one microbial population; or (c) a communal or cooperative resistance of two or more microbial species, taxonomic classification above the level of species, taxonomic classification below the level of species, strain, Gram stain classification, clone, or phenotype present or suspected to be present in the at least one microbial population.

607. The method according to any one of embodiments 601-606, wherein the mass spectrometer is selected from the group consisting of: an electrospray mass spectrometer, a desorption electrospray ionization mass spectrometer, a time-of-flight mass spectrometer, a quadrupole mass spectrometer, a triple quadrupole mass spectrometer, a magnetic sector mass spectrometer, an ion trap mass spectrometer, a quadrupole trap mass spectrometer, an orbitrap mass spectrometer, a gas chromatograph mass spectrometer, a matrix-assisted laser desorption/ionization (“MALDI”) mass spectrometer, a Time-of-Flight Secondary Ion Mass Spectrometry (“TOF-SIMS”) mass spectrometer, an ion mobility mass spectrometer, a plasma chromatograph, an inductively-coupled plasma mass spectrometer, a mass cytometer, an accelerator mass spectrometer, a Fourier transform mass spectrometer, a Fourier-transform ion cyclotron resonance mass spectrometer, a mass spectrometer using an ambient ionization method such as direct analysis in real time, a mass spectrometer using surface acoustic wave nebulization or another nebulization technique, a mass spectrometer using Rapid Evaporative Ionization Mass Spectrometry, or another type of mass spectrometer.

608. The method according to any one of embodiments 601-607, wherein the one or more mass spectra is selected from the group consisting of a parent ion scan, tandem ion scan, or MSⁿ scan, and/or is produced by means of data-independent precursor ion selection, data-dependent precursor ion selection, multiple reaction monitoring (“MRM”), selected reaction monitoring (“SRM”), consecutive reaction monitoring (“CRM”), parallel reaction monitoring (“PRM”), or Precursor Acquisition Independent From Ion Count (“PAcIFIC”).

609. The method according to any one of embodiments 601-608, wherein at least one concentration of deuterium is equal to about the natural abundance of deuterium corresponding to an increase above natural abundance of 0%, or greater than the natural abundance of deuterium by more than 20%, about 20%, between 20% and 10%, about 10%, between 10% and 8%, about 8%, between 8% and 5%, about 5%, between 5% and 3%, about 3%, or less than 3%.

610. The method according to any one of embodiments 601-609, comprising incubating all or a portion of the sample over one or more time intervals, where incubation is at a temperature less than 25° C., about 25° C., between 25° C. and 30° C., about 30° C., between 30° C. and 35° C., about 35° C., between 35° C. and 37° C., about 37° C., or greater than 37° C.; and
wherein one or more of the one or more time intervals is less than 30 minutes, about 30 minutes, between 30 minutes and 1 hour, about 1 hour, between 1 and 2 hours, about 2 hours, between 2 and 3 hours, about 3 hours, between 3 and 4 hours, about 4 hours, between 4 and 5 hours, about 5 hours, between 5 and 6 hours, about 6 hours, between 6 and 7 hours, about 7 hours, between 7 and 10 hours, about 10 hours, between 10 and 16 hours, about 16 hours, between 16 and 24 hours, about 24 hours, between 24 and 48 hours, about 48 hours, or greater than 48 hours.

611. The method according to any one of embodiments 601-610, wherein at least one deuterated or undeuterated ion is a product of at least one of a membrane lipid, a phospholipid, a lipid A, a lipopolysaccharide, a phosphatidylcholine, a phosphatidylethanolamine, a phosphoglycerol, a cardiolipin, a lipoteichoic acid, a sphingolipid, a sterol, or a member of phospholipid species PE29:1, PE29:2, PE29:3, PE30:1, PE30:2, PE30:3, PE31:1, PE31:2, PE31:3, PE32:1, PE32:2, PE32:3, PE33:1, PE33:2, PE33:3, PE34:1, PE34:2, PE34:3, PE35:1, PE35:2, PE35:3, PG30:1, PG30:2, PG30:3, PG31:1, PG31:2, PG31:3, PG32:1, PG32:2, PG32:3, PG33:1, PG33:2, PG33:3, PG34:1, PG34:2, PG34:3, PG35:1, PG35:2, PG35:3, PG36:1, PG36:2, or PG36:3.

612. The method according to any one of embodiments 601-611, further comprising extracting lipids for analysis from the sample by a method selected from the group consisting of: the fast lipid assay test method, the BACLIB method considered as a lipid extraction method, the Caroff method, the Bligh-Dyer method, the Folch method, the hot phenol method, two-phase extraction, or the TAO method.

613. The method according to any one of embodiments 601-612, wherein applying the portion extracted from the liquid solution containing the sample to the mass spectrometer comprises:
placing the liquid solution from the environment in contact with at least one surface, wherein the at least one surface is optionally made of steel or stainless steel;
optionally enclosing the steel surface to facilitate control of evaporation of the liquid solution;
optionally adding a solution of citric acid and sodium citrate or another wet or dry buffer or acidic composition to the steel surface or to the liquid solution;
optionally heating the liquid solution on the steel surface;
optionally washing the steel surface;
optionally drying the steel surface; and
optionally applying a composition comprising at least one solvent or substance having the property of acting as a MALDI matrix.

614. The method according to embodiment 613, wherein the liquid solution is heated for less than 5 minutes, between 5 minutes and 15 minutes, about 15 minutes, about 20 minutes, about 25 minutes, about 30 minutes, or longer than 30 minutes.

615. The method according to embodiment 614, wherein the heating comprises heating the liquid solution in contact with the steel surface to less than 80° C., about 80° C., between 80° C. and 95° C., about 95° C., about 100° C., about 110° C., about 121° C., about 125° C., about 130° C., about 135° C., or more than 135° C.

616. The method according to any one of embodiments 601-615, wherein lipids are extracted for analysis from at least one microbial population or at least one environment containing at least one sample by a method comprising:
placing a quantity of liquid from the environment in a separation column containing at least one solvent and/or a solvent gradient;
extracting lipids from at least one organism into solution in the column;
eluting lipids from the column into a mass spectrometer.

617. The method according to any one of embodiments 601-616, wherein the mass spectrometer optionally is in a negative ionization mode, and at least one spectrum is collected with a lowest m/z of less than 100, about 100, about 300, about 400, about 600, about 700, about 800, about 1000, or greater than 1000 m/z and a highest m/z of less than 200, about 200, about 300, about 400, about 600, about 700, about 800, about 900, about 1000, about 1200, about 1500, about 1800, about 2000, about 2200, about 2400, about 2500 or greater than 2500 m/z.

618. The method according to any one of embodiments 601-617, wherein the liquid solution is applied to another analytical instrument operating by means selected from the group consisting of: laser induced fluorescence spectroscopy, atomic absorption spectroscopy, atomic emission spectroscopy, flame emission spectroscopy, acoustic resonance spectroscopy, cavity ring down spectroscopy, circular dichroism spectroscopy, Raman spectroscopy, surface enhanced Raman spectroscopy, coherent Raman spectroscopy, vibrational Raman spectroscopy, spontaneous Raman spectroscopy, enhanced Raman spectroscopy, Stokes Raman spectroscopy, Anti-Stokes Raman spectroscopy, enhanced Raman spectroscopy, surface-enhanced Raman spectroscopy, stimulated Raman spectroscopy, inverse Raman spectroscopy, coherent anti-Stokes Raman spectroscopy, resonance Raman spectroscopy, transmission Raman spectroscopy, Raman spectroscopy of micro-cavity substrates, Raman spectroscopy combined with time-correlated photon counting, cold vapor atomic fluorescence spectroscopy, nuclear magnetic resonance spectroscopy, electrical impedance spectroscopy, electron phenomenological spectroscopy, electron paramagnetic resonance spectroscopy, Fourier-transform spectroscopy, laser-induced breakdown spectroscopy, photoacoustic spectroscopy, photoemission spectroscopy, photothermal spectroscopy, spectrophotometry, vibrational circular dichroism spectroscopy, gamma spectroscopy, NMR spectroscopy, flow cytometry, or some other type of spectroscopy; or by means of a scintillation detector, scintillation counter, Geiger counter, ionization chamber, gaseous ionization detector, or other radiation detector.

619. The method according to any one of embodiments 601-618, wherein additionally the presence, quantity, taxon, or other property of the at least one microbial population in the sample is determined using at least one spectrum produced by the mass spectrometer or other analytical instrument;
wherein the taxon or the other property of the at least one microbial population is a bacterial species; a yeast or fungal species; a microbial species other than bacteria, a yeast, or a fungi; a microbial strain; a microbial phenotype of resistance, intermediate resistance, or susceptibility to one or more antimicrobial agent, antimicrobial class, or antimicrobial combination; a microbial taxonomic classification above the level of species; a microbial taxonomic classification below the level of species, a Gram stain classification, or a clone; and
wherein optionally, the presence, quantity, taxon, or other property of the at least one microbial population in the sample is determined by the BACLIB method.

620. The method according to any one of embodiments 601-619, wherein the sample is derived from a culture plate colony or smear, a broth culture sample, a blood culture sample, a sample from a biofluid, a clinical sample, a nonclinical sample, an environmental sample, a veterinary sample, an agricultural sample, a food or food safety sample, an industrial sample, a process control sample, a forensic sample, a biofluid from a human or non-human source, a urine specimen, a blood sample, a sample incubated in a blood bottle, sputum, a sample obtained from sputum, urine, feces, wound effluent, mucus, buccal swab, nasal swab, vaginal swab, nipple aspirate, sweat, saliva, semen or ejaculate, synovial fluid, bronchoalveolar lavage, endotracheal aspirate, tears, a urinary catheter sample, a culture plate, or another clinical or medical sample, or another human or mammalian material.

621. The method according to any one of embodiments 601-620, wherein the liquid solution applied to the mass spectrometer or other analytical instrument has a volume of less than about 0.2 μL, about 0.2 μL, about 0.3 μL, about 0.4 μL, about 0.5 μL, about 1.0 μL, about 1.5 μL, about 2 μL, about 3 μL, about 4 μL, about 6 μL, about 8 μL, about 10 μL, about 20 μL, or greater than 10 μL.

622. The method according to any one of embodiments 601-621, wherein the detection or measurement of growth of at least one microbial population is used to diagnose a human or non-human disease, to predict the progress of the human or non-human disease, or to estimate the efficacy of a treatment for the human or non-human disease.

623. A kit for use in practicing the method according any one of embodiments 601-622, the kit comprising:
an analytical instrument;
a plate, wherein the plate comprises a material disposed on at least a portion of at least one surface of the plate;
a gasket, wherein the gasket comprises at least one hole having a shape and a size configured for placement over and alignment with at least one structured spot on the plate;
a solvent, wherein the solvent comprises any one or more of a buffer solution, an acidic solution, and a solid to which a liquid can be added to form a buffer or an acidic solution;
an enclosure, wherein the enclosure comprises a holder base, optionally the holder base comprises a reservoir, and a lid; and
a computing device or system comprising a software configured to receive or display spectroscopic data.

624. A method of generating a deuterated lipid or deuterated lipid mimetic, comprising:
obtaining a bacterial strain that expresses one or more non-endogenous lipid biosynthesis enzymes; expresses one or more endogenous lipid biosynthesis enzymes; or has modified regulation of one or more endogenous lipid biosynthesis enzymes;
passing the bacterial strain one or more times through one or more environments with deuterated solution;
subjecting the strain to conditions suitable for production of a lipid composition, in an environment with deuterated solution.

625. The method according to embodiment 624, wherein the deuterated lipid or deuterated lipid mimetic is a lipooligosaccharide/lipid A-based mimetic, a deuterated PC, PE, PG, LPS, LA, SP, LTA, or ST or other lipid or a deuterated mimetic of a PC, PE, PG, LPS, LA, SP, LTA, or ST or other lipid or lipid mimetic.

626. A method of enhancing or suppressing an immune response in a subject, comprising:
obtaining a deuterated lipid or deuterated lipid mimetic according to embodiments 624 or 625, or a lipid or lipid mimetic described in embodiment 625;
administering to the subject an effective amount of the deuterated lipid or deuterated lipid mimetic, alone, as part of an emulsion, suspension, or other mixture, and/or in combination with other therapeutic or other agents, and/or as an anti-inflammatory agent, adjuvant, part of a substance acting as an anti-inflammatory agent or adjuvant, or otherwise a component of a vaccine. administering to the subject an effective amount of the deuterated lipid or deuterated lipid mimetic.

627. A method of treating or preventing inflammation and/or sepsis in a subject, comprising:
administering to the subject an effective amount of at least one immunomodulation lipid, wherein the immunomodulation lipid is a lipid described in embodiment 625.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Patent Application No. 62/905,361, filed Sep. 24, 2019, and U.S. Patent Application No. 62/956,438, filed Jan. 2, 2020, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for detecting growth of at least one microbial population and/or growth of at least one non-microbial cellular population in a sample, comprising:
(a) incubating a sample containing or suspected to contain the at least one microbial population in an environment, wherein the environment comprises deuterium in a liquid solution at a concentration that is above a natural abundance of the deuterium in the liquid solution;
(b) extracting all or a portion of a substance from the environment and applying the all or a portion of the substance to a mass spectrometer;
(c) producing one or more mass spectra using the mass spectrometer; and
(d) detecting growth of the at least one microbial population in the sample by identifying one or more peaks representing any one or more of: (i) the presence of at least one deuterated ion; (ii) the presence of at least one deuterated ion and one undeuterated ion; (iii) the presence of at least one pattern of deuterated ions; (iv) the presence of at least one pattern of deuterated ions and at least one pattern of undeuterated ions; or (v) the presence of at least one pattern of deuterated and undeuterated ions.

2. The method of claim 1, further comprising measuring a relative or absolute degree of growth of the at least one microbial population in the sample as (1) the ratio of at least one deuterated ion to at least one undeuterated ion and/or the (2) ratio of at least one deuterated ion pattern to at least one undeuterated ion pattern.

3. The method of claim 1, wherein the sample comprises one or more subsamples and wherein the method further comprises, prior to or during the incubating in (a):
exposing one or more of the subsamples to at least one chemical agent at one or more concentration; and
identifying a growth inhibitory effect or a growth enabling and/or acceleration effect of the at least one chemical agent as: (1) a minimum concentration of the at least one chemical agent at which growth is not observed; (2) a minimum concentration of the at least one chemical agent calculated from growth measurements at one or more concentrations and one or more points in time; or (3) a growth rate calculated from growth measurements at one or more concentrations and one or more points in time.

4. (canceled)

5. The method according to claim 3, or wherein the at least one chemical agent is an antimicrobial agent, antimicrobial class, or antimicrobial combination, and the detected growth is used to determine antimicrobial susceptibility or resistance of at least one of (a)-(c):
(a) the at least one microbial population; (b) at least one microbial species, taxonomic classification above the level of species, taxonomic classification below the level of species, strain, Gram stain classification, clone, or phenotype present or suspected to be present in the at least one microbial population; or (c) a communal or cooperative resistance of two or more microbial species, taxonomic classification above the level of species, taxonomic classification below the level of species, strain, Gram stain classification, clone, or phenotype present or suspected to be present in the at least one microbial population.

6. (canceled)

7. The method according to claim 1, wherein (a) the mass spectrometer is selected from the group consisting of: an electrospray mass spectrometer, a desorption electrospray ionization mass spectrometer, a time-of-flight mass spectrometer, a quadrupole mass spectrometer, a triple quadrupole mass spectrometer, a magnetic sector mass spectrometer, an ion trap mass spectrometer, a quadrupole trap mass spectrometer, an orbitrap mass spectrometer, a gas chromatograph mass spectrometer, a matrix-assisted laser desorption/ionization (“MALDI”) mass spectrometer, a Time-of-Flight Secondary Ion Mass Spectrometry (“TOF-SIMS”) mass spectrometer, an ion mobility mass spectrometer, a plasma chromatograph, an inductively-coupled plasma mass spectrometer, a mass cytometer, an accelerator mass spectrometer, a Fourier transform mass spectrometer, a Fourier-transform ion cyclotron resonance mass spectrometer, a mass spectrometer using an ambient ionization method such as direct analysis in real time, a mass spectrometer using surface acoustic wave nebulization or another nebulization technique, a mass spectrometer using Rapid Evaporative Ionization Mass Spectrometry, and another type of mass spectrometer; or (b) the one or more mass spectra is selected from the group consisting of a parent ion scan, tandem ion scan, and MSⁿ scan, and/or is produced by means of data-independent precursor ion selection, data-dependent precursor ion selection, multiple reaction monitoring (“MRM”), selected reaction monitoring (“SRM”), consecutive reaction monitoring (“CRM”), parallel reaction monitoring (“PRM”), or Precursor Acquisition Independent From Ion Count (“PAcIFIC”).

8. (canceled)

9. The method according to claim 1, wherein at least one concentration of deuterium is equal to about the natural abundance of deuterium corresponding to an increase above natural abundance of 0%, or greater than the natural abundance of deuterium by more than 20%, about 20%, between 20% and 10%, about 10%, between 10% and 8%, about 8%, between 8% and 5%, about 5%, between 5% and 3%, about 3%, or less than 3%.

10. The method according to claim 1, further comprising incubating all or a portion of the sample over one or more time intervals, where incubation is at a temperature less than 25° C., about 25° C., between 25° C. and 30° C., about 30° C., between 30° C. and 35° C., about 35° C., between 35° C. and 37° C., about 37° C., or greater than 37° C.; and
wherein one or more of the one or more time intervals is less than 30 minutes, about 30 minutes, between 30 minutes and 1 hour, about 1 hour, between 1 and 2 hours, about 2 hours, between 2 and 3 hours, about 3 hours, between 3 and 4 hours, about 4 hours, between 4 and 5 hours, about 5 hours, between 5 and 6 hours, about 6 hours, between 6 and 7 hours, about 7 hours, between 7 and 10 hours, about 10 hours, between 10 and 16 hours, about 16 hours, between 16 and 24 hours, about 24 hours, between 24 and 48 hours, about 48 hours, or greater than 48 hours.

11. The method according to claim 1, wherein at least one deuterated or undeuterated ion is a product of at least one of a membrane lipid, a phospholipid, a lipid A, a lipopolysaccharide, a phosphatidylcholine, a phosphatidylethanolamine, a phosphoglycerol, a cardiolipin, a lipoteichoic acid, a sphingolipid, a sterol, or a member of phospholipid species PE29:1, PE29:2, PE29:3, PE30:1, PE30:2, PE30:3, PE31:1, PE31:2, PE31:3, PE32:1, PE32:2, PE32:3, PE33:1, PE33:2, PE33:3, PE34:1, PE34:2, PE34:3, PE35:1, PE35:2, PE35:3, PG30:1, PG30:2, PG30:3, PG31:1, PG31:2, PG31:3, PG32:1, PG32:2, PG32:3, PG33:1, PG33:2, PG33:3, PG34:1, PG34:2, PG34:3, PG35:1, PG35:2, PG35:3, PG36:1, PG36:2, or PG36:3.

12. The method according to claim 1, further comprising extracting lipids for analysis from the sample by a method selected from the group consisting of: the fast lipid assay test method, the BACLIB method considered as a lipid extraction method, the Caroff method, the Bligh-Dyer method, the Folch method, the hot phenol method, two-phase extraction, and the TAO method.

13. The method according to claim 1, wherein applying the portion extracted from the liquid solution containing the sample to the mass spectrometer comprises:
placing the liquid solution from the environment in contact with at least one surface, wherein the at least one surface is optionally made of steel or stainless steel;
optionally enclosing the steel surface to facilitate control of evaporation of the liquid solution;
optionally adding a solution of citric acid and sodium citrate or another wet or dry buffer or acidic composition to the steel surface or to the liquid solution;
optionally heating the liquid solution on the steel surface;
optionally washing the steel surface;
optionally drying the steel surface; and
optionally applying a composition comprising at least one solvent or substance having the property of acting as a MALDI matrix.

14. The method according to claim 13, wherein the liquid solution is heated for less than 5 minutes, between 5 minutes and 15 minutes, about 15 minutes, about 20 minutes, about 25 minutes, about 30 minutes, or longer than 30 minutes; and
the liquid solution is heated in contact with the steel surface to less than 80° C., about 80° C., between 80° C. and 95° C., about 95° C., about 100° C., about 110° C., about 121° C., about 125° C., about 130° C., about 135° C., or more than 135° C.

15. (canceled)

16. The method according to claim 1, wherein lipids are extracted for analysis from at least one microbial population or at least one environment containing at least one sample by a method comprising:
placing a quantity of liquid from the environment in a separation column containing at least one solvent and/or a solvent gradient;
extracting lipids from at least one organism into solution in the column; and
eluting lipids from the column into a mass spectrometer.

17. The method according to claim 1, wherein the mass spectrometer optionally is in a negative ionization mode, and at least one spectrum is collected with a lowest m/z of less than 100, about 100, about 300, about 400, about 600, about 700, about 800, about 1000, or greater than 1000 m/z and a highest m/z of less than 200, about 200, about 300, about 400, about 600, about 700, about 800, about 900, about 1000, about 1200, about 1500, about 1800, about 2000, about 2200, about 2400, about 2500, or greater than 2500 m/z.

18. (canceled)

19. The method according to claim 1, wherein additionally the presence, quantity, taxon, or other property of the at least one microbial population in the sample is determined using at least one spectrum produced by the mass spectrometer or other analytical instrument;
wherein the taxon or the other property of the at least one microbial population is a bacterial species; a yeast or fungal species; a microbial species other than bacteria, a yeast, or a fungi; a microbial strain; a microbial phenotype of resistance, intermediate resistance, or susceptibility to one or more antimicrobial agent, antimicrobial class, or antimicrobial combination; a microbial taxonomic classification above the level of species; a microbial taxonomic classification below the level of species, a Gram stain classification, or a clone; and
wherein optionally, the presence, quantity, taxon, or other property of the at least one microbial population in the sample is determined by the BACLIB method.

20. The method according to claim 1, wherein the sample is derived from a culture plate colony or smear, a broth culture sample, a blood culture sample, a sample from a biofluid, a clinical sample, a nonclinical sample, an environmental sample, a veterinary sample, an agricultural sample, a food or food safety sample, an industrial sample, a process control sample, a forensic sample, a biofluid from a human or non-human source, a urine specimen, a blood sample, a sample incubated in a blood bottle, sputum, a sample obtained from sputum, urine, feces, wound effluent, mucus, buccal swab, nasal swab, vaginal swab, nipple aspirate, sweat, saliva, semen or ejaculate, synovial fluid, bronchoalveolar lavage, endotracheal aspirate, tears, a urinary catheter sample, a culture plate, or another clinical or medical sample, or another human or mammalian material.

21. The method according to claim 1, wherein the liquid solution applied to the mass spectrometer or other analytical instrument has a volume of less than about 0.2 μL, about 0.2 μL, about 0.3 μL, about 0.4 μL, about 0.5 μL, about 1.0 μL, about 1.5 μL, about 2 μL, about 3 μL, about 4 μL, about 6 μL, about 8 μL, about 10 μL, about 20 μL, or greater than 10 μL.

22. The method according to claim 1, wherein the detection or measurement of growth of at least one microbial population is used to diagnose a human or non-human disease, to predict the progress of the human or non-human disease, or to estimate the efficacy of a treatment for the human or non-human disease.

23. A kit for use in practicing the method according to claim 1, the kit comprising:
an analytical instrument;
a plate, wherein the plate comprises a material disposed on at least a portion of at least one surface of the plate;
a gasket, wherein the gasket comprises at least one hole having a shape and a size configured for placement over and alignment with at least one structured spot on the plate;
a solvent, wherein the solvent comprises any one or more of a buffer solution, an acidic solution, and a solid to which a liquid can be added to form a buffer or an acidic solution;
an enclosure, wherein the enclosure comprises a holder base, optionally the holder base comprises a reservoir, and a lid; and
a computing device or system comprising a software configured to receive or display spectroscopic data.

24.-25. (canceled)

26. A method of enhancing or suppressing an immune response in a subject, and/or treating or preventing inflammation and/or sepsis in the subject, comprising:
administering to the subject an effective amount of a deuterated lipid or deuterated lipid mimetic, alone, as part of an emulsion, suspension, or other mixture, and/or in combination with other therapeutic or other agents, and/or as an anti-inflammatory agent, adjuvant, part of a substance acting as an anti-inflammatory agent or adjuvant, or otherwise a component of a vaccine.

27. (canceled)

28. A method for detecting growth of at least one microbial population and/or growth of at least one non-microbial cellular population in a sample, comprising:
(a) incubating a sample containing or suspected to contain the at least one microbial population in an environment, wherein the environment comprises deuterium in a liquid solution at a concentration that is above a natural abundance of the deuterium in the liquid solution; and
(b) extracting all or a portion of a substance from the environment and applying the all or a portion of the substance to an analytical instrument operating by: laser induced fluorescence spectroscopy, atomic absorption spectroscopy, atomic emission spectroscopy, flame emission spectroscopy, acoustic resonance spectroscopy, cavity ring down spectroscopy, circular dichroism spectroscopy, Raman spectroscopy, surface enhanced Raman spectroscopy, coherent Raman spectroscopy, vibrational Raman spectroscopy, spontaneous Raman spectroscopy, enhanced Raman spectroscopy, Stokes Raman spectroscopy, Anti-Stokes Raman spectroscopy, enhanced Raman spectroscopy, surface-enhanced Raman spectroscopy, stimulated Raman spectroscopy, inverse Raman spectroscopy, coherent anti-Stokes Raman spectroscopy, resonance Raman spectroscopy, transmission Raman spectroscopy, Raman spectroscopy of micro-cavity substrates, Raman spectroscopy combined with time-correlated photon counting, cold vapor atomic fluorescence spectroscopy, nuclear magnetic resonance spectroscopy, electrical impedance spectroscopy, electron phenomenological spectroscopy, electron paramagnetic resonance spectroscopy, Fourier-transform spectroscopy, laser-induced breakdown spectroscopy, photoacoustic spectroscopy, photoemission spectroscopy, photothermal spectroscopy, spectrophotometry, vibrational circular dichroism spectroscopy, gamma spectroscopy, NMR spectroscopy, flow cytometry, or other type of spectroscopy; or by a scintillation detector, scintillation counter, Geiger counter, ionization chamber, gaseous ionization detector, or other radiation detector.
